(19) United States
(12) Patent Application Publication
(10) Pub. No.: US 2024/0156886 A1
(43) Pub. Date: May 16, 2024

(54) GOS PRE-CONDITIONING LACTOBACILLUS STRAINS AND GOS IN FINAL FORMULATION

(71) Applicant: SOCIETE DES PRODUITS NESTLE S.A., Vevey (CH)

(72) Inventors: FLORAC DE BRUYN, Lausanne (CH); BERTRAND BOURQUI, Bern (CH); GUENOLEE ELIANE MARIE PRIOULT, Morristown, NJ (US); STEPHANE DUBOUX, St-Prex (CH)

(21) Appl. No.: 18/549,669

(22) PCT Filed: Mar. 11, 2022
(86) PCT No.: PCT/EP2022/056284

§ 371 Date: Sep. 8, 2023

(30) Foreign Application Priority Data
Mar. 12, 2021 (EP) 21162403.6

(51) Int. Cl. A61K 35/747; C12N 1/20; C12N 1/38; C12R 1/23

(52) U.S. Cl. CPC A61K 35/747; C12N 1/20; C12N 1/38; C12N 2500/34; C12R 2001/23

(57) ABSTRACT

The invention herein relates generally to enhancing the survival and activity of probiotic Lactobacillus strains in mammals. Lactobacillus strain comprising a LacS transporter sequence having a sequence identity of at least 70% to SEQ ID NO: 1, and/or comprising a GH42 B-gal sequence, wherein the Lactobacillus strain is not a Lactobacillus reuteri strain. The invention comprises methods for cultivating and manufacturing probiotic Lactobacillus strains, and products containing such strains. In more detail the GC present invention relates to a composition comprising probiotic Lactobacillus strains, obtained by a step of growing the bacteria in a medium comprising galacto-oligosaccharides (GOS) (“pre-conditioning”), and administering said pre-conditioned bacteria together with GOS in the final product. The inventors have found that such method induces a boost in the beneficial effects of the probiotic bacteria, such as survival in the gastrointestinal tract.

### TECHNICAL FIELD

The invention herein relates generally to enhancing the survival and activity of probiotic Lactobacillus strains in mammals. Lactobacillus strain comprising a LacS transporter sequence having a sequence identity of at least 70% to SEQ ID NO: 1, and/or comprising a GH42 B-gal sequence, wherein the Lactobacillus strain is not a Lactobacillus reuteri strain. The invention comprises methods for cultivating and manufacturing probiotic Lactobacillus strains, and products containing such strains. In more detail the present invention relates to a composition comprising probiotic Lactobacillus strains, obtained by a step of growing the bacteria in a medium comprising galacto-oligosaccharides (GOS) (“pre-conditioning”), and administering said pre-conditioned bacteria together with GOS in the final product. The inventors have found that such method induces a boost in the beneficial effects of the probiotic bacteria, such as survival and metabolic activity in the gastrointestinal tract.

### BACKGROUND

Probiotics are defined as “live microorganisms that, when administered in adequate amounts, confer a health benefit on the host.” This is the widely accepted scientific definition around the world (see e.g. Hill et al. 2014; Nat. Rev. Gastro. & Hepathology, Vol 11). Probiotic products (which is usually dietary supplements or foods) may be recommended for different conditions or symptoms an individual is experiencing. Lactic acid producing bacteria, such as Lactobacilli, are commonly used as probiotics in various types of foods, for example yoghurt. Growth and colonization of harmful microorganisms can be prevented by such lactic acid producing bacteria through their own colonization inside the intestinal system, through competition of available nutrients and/or through production of specific substances, such as hydrogen peroxide, bacteriocins and/or organic acids, including lactic and acetic acid, that lowers the intestinal pH. It is well established that interactions between host and gut microbes are fundamental to health and well-being of the host. Intestinal microbiota generates metabolites that provide the host with nutrients but may also be involved in the immune response and in regulation and development of the host's immune system as well as reducing inflammation and preventing allergic responses.

Prebiotics are compounds that induce the growth or activity of beneficial microorganisms such as bacteria and fungi by selectively stimulating their growth and/or activity. In the gastrointestinal tract, prebiotics can therefore alter the composition of the gut microbiome. Dietary prebiotics are typically nondigestible (see e.g., https://en.wilepedia.orerwikeDietary fiber) compounds that pass undigested through the upper part of the gastrointestinal tract and stimulate the growth or activity of advantageous bacteria that colonize the large bowel by acting as their substrate. Various compounds have been tested to determine their function as prebiotics. Fructo-oligosaccharides (FOS), galacto-oligosaccharides (GOS), and trans-galacto-oligosaccharides (TOS) are the most common prebiotics. Consuming certain prebiotics can thus improve immunity functions by increasing the population of microorganisms associated with human health, and animal and human studies have shown that prebiotics can decrease the population of harmful bacteria. Prebiotics are thus ingredients, providing health benefits when fermented by the native microflora in the intestine of a subject or by probiotic bacteria ingested simultaneously with the prebiotics.

Combining particular types of prebiotics and probiotics is generally known to give rise to certain synergistic effects. In particular, the effect of combined prebiotic galacto-oligosaccharides (GOS) and probiotic microorganisms, such as Lactobacillus and Bifidobacteria, has been documented (see e.g. YUTAKA KANAMORI, M D et al., Digestive Diseases and Sciences, Vol. 46, No. 9 (September 2001), pp. 2010-2016 (®2001)). It has been demonstrated that the synbiotic effect between prebiotics and probiotics is due to the consumption of the prebiotic by the probiotic within the gastrointestinal tract of the subject.

The manufacturing procedure of probiotic lactic acid producing bacteria is typically standardized and involves a step of fermenting the bacteria in a growth medium comprising a carbohydrate source, such as a sugar, for example glucose, fructose, sucrose, lactose or dextrose. Following the fermentation, the probiotic bacteria are usually protected and frozen or freeze-dried and packaged into a finished product.

The ingestion of probiotic lactic acid bacteria has various effects in vivo. One such important effect is the bioavailability of essential minerals, which can be significantly influenced by the presence of such probiotic lactic acid bacteria. Although the content of minerals in the body depends primarily on their supply from food intake and they are absorbed mainly in the GI tract, mere provision of minerals via food intake does not necessarily lead to a sufficient mineral bioavailability. The main factors affecting mineral bioavailability are the content of minerals in the food, synergistic and antagonistic interactions between minerals in the food and in the GI tract, the presence of complexing or chelating compounds in the food, and the health state of the organism and its age. Also, the intestinal microflora, probiotics and prebiotics significantly influence the bioavailability of minerals and can thereby increase or decrease the absorption of such minerals.

The ingestion of probiotics in general and of lactic acid bacteria in particular, has also been associated with numerous functions such as the function for the digestive tract (digestive function and/or nutrient absorption), the function of the immune system and the overall growth and development of infants, and related to the promotion of a healthy microbiota in a subject.

In view of the above, it is therefore an object of the current invention to provide more efficient means, methods and therapies to promote a healthy microbiota in a subject, promote the digestive functions, immune functions and/or overall growth and development. Moreover, it is an object of improving the probiotic effects of probiotic microorganisms, such as Lactobacilli, when administering such probiotics to a subject or a patient in need thereof.

These objects are solved by the current invention as addressed below.

### SUMMARY OF THE INVENTION

The present invention is defined below and in the independent claims. Further embodiments of the invention are defined in the specification and in the dependent claims.

In a first aspect, the invention provides a method for preparing a composition comprising a pre-conditioned probiotic Lactobacillus strain and GOS, characterized in that the method comprises the steps:
a) pre-conditioning of a probiotic Lactobacillus strain following the steps:
   i) cultivating a probiotic Lactobacillus strain in the presence of galacto-oligosaccharide (GOS) in a growth medium, thereby pre-conditioning the probiotic Lactobacillus strain; and
   ii) optionally harvesting the pre-conditioned probiotic Lactobacillus strain from the growth medium;
b) preparing a composition comprising the pre-conditioned probiotic Lactobacillus strain obtained from step a)i) or aii) and adding GOS to the composition,
wherein the probiotic Lactobacillus strain is provided from at least one Lactobacillus strain comprising a LacS transporter sequence having a sequence identity of at least 65% or preferably 70% to SEQ ID NO: 1, and/or comprising a GH42 B-gal sequence, wherein the Lactobacillus strain is not a Lactobacillus reuteri strain.

In the inventive method, the GOS may be added in step a)i) to the growth medium in an amount of at least 0.2 wt %, preferably at least 0.5 wt %, even more preferably at least 0.75 wt % of GOS in the growth medium, and preferably at most 8 wt % of GOS, such as at most 7 wt % GOS, at most 6 wt % GOS, or even at most 5 wt % GOS, more preferably at most 4 wt % of GOS or even at most 3% GOS at most 2 wt % of GOS, or at most 1 wt % of GOS in the growth medium. Any combination of these upper and lower ranges is encompassed herewith. More preferably, GOS is added to the growth medium in a range of 0.2 wt % to 8 wt % or even 0.2 wt % to 7 wt %, or 0.2 wt % to 6 wt %, more preferably in a range of 0.5 wt % to 8 wt % or even 0.5 wt % to 7 wt %, or 0.5 wt % to 6 wt %, even more preferably in a range of 0.75 wt % to 8 wt % or even 0.75 wt % to 7 wt %, or 0.75 wt % to 6 wt %, and most preferably in a range of 0.75 wt % to 7 wt %, in a range of 0.75 wt % to 6 wt % or even in a range of 0.75 wt % to 5 wt %, such as a range of 0.75 wt % to 5 wt % or 0.75 wt % to 4 wt %, preferably calculated on basis of the growth medium (% w/w). Addition of GOS may occur preferably at the start, in the middle or at the end of cultivation of a probiotic Lactobacillus strain in step a)i), and/or at once, stepwise or continuously.

Moreover, GOS may be preferably added in step b) to the pre-conditioned probiotic Lactobacillus strain obtained from step a) in an amount of at least 1 or 2 g per daily dose, preferably at least 3 g per daily dose, likewise preferably between 2 to 12 g per daily dose, or between 2 to 10 g per daily dose, between 2 to 8 g per daily dose, or between 2 to 7 g per daily dose, more preferably between 3 to 12 g per daily dose, likewise more preferably between 3 and 10 g per daily dose, such as between 3 to 8 g per daily dose, between 3 to 7 g per daily dose, between 3 to 6 g per daily dose, or about 2, 3, 4, 5, 6, 7, 8, 9, 10, 11, or 12 g per daily dose or any range formed thereby.

Furthermore, the pre-conditioned probiotic Lactobacillus strain obtained in step b) from step a)i) or a)ii) is preferably present in the composition in an amount of between 10³ cfu to 10¹² cfu per daily dose, likewise preferably in an amount of between 10⁴ cfu to 10¹¹ cfu per daily dose, more preferably in an amount of between 10⁵ cfu to 10⁹ cfu per daily dose, even more preferably in an amount of between 10⁶ cfu to 10⁹ cfu per daily dose or in an amount of 10⁸ cfu to 10¹⁰ cfu per daily dose, most preferably in an amount of around 10⁸ total cfu per daily dose, including an amount of 10⁷ to 10⁹ or 10⁸ to 10⁹ cfu per daily dose.

Moreover, the GOS added to the growth medium in step a)i) of the inventive method and/or to the composition comprising the pre-conditioned probiotic Lactobacillus strain prepared in step b) of the inventive method preferably may have a degree of polymerization (DP) of 3-8. The cultivation in step a)i) of the inventive method may occur over a period of 5 to 18 hours, preferably 7 to 14 hours, and more preferably 10 to 13 hours, and even more preferably about 12 hours. According to a preferred embodiment, cultivation in step a)i) of the inventive method occurs at a temperature of between 25 to 45° C., preferably between 30 and 40° C., most preferably at a temperature of about 37° C. The pH during pre-conditioning of probiotic Lactobacillus strain during cultivation step a) may be either not controlled or set to a constant pH value throughout the cultivation step a)i), typically to be monitored and adjusted during cultivation step a). Accordingly, the pH may be between 3.0 and 7.0, particularly when pH is not controlled during pre-conditioning of probiotic Lactobacillus strain. Alternatively, if pH is controlled during cultivation step a)i), pH may be adjusted to a range of about 5.0 to 7.0, e.g., about 5.0 to 6.0 or about 6.0 to 7.0, preferably pH is adjusted to a range of about 6.0 to 7.0, more preferably to a range of about 6.6±0.4, most preferably to a range of about 6.6±0.2.

It is added, that the inventive method for preparing a pre-conditioned probiotic Lactobacillus strain may comprise a further step a)iii) of washing the pre-conditioned probiotic Lactobacillus strain after harvesting step a)ii), preferably to remove trace amounts of growth medium. The inventive method may also comprise a further step a)iv) of drying the harvested pre-conditioned probiotic Lactobacillus strain, step a)iv) either carried out after washing step a)iii) or after harvesting step a)ii). The drying step a)iv) may be carried out using spray-drying, fluid bed drying, air convective drying, atmospheric drying, roller drying or freeze drying, lyophilizing, and more preferably spray-drying or freeze drying. In case of need the drying step may be preceded by treating the harvested pre-conditioned probiotic Lactobacillus strain with a protectant and/or a carrier. Alternatively, or additionally, the pre-conditioned Lactobacillus strain obtained after any of steps a)i, a)ii) or a)iii) or a)iv) may be encapsulated for further processing, preferably prior to the drying step a)iv). The Lactobacillus strain is preferably a Lactobacillus acidophilus (L. acidophilus) strain, more preferably Lactobacillus acidophilus (L. acidophilus) strain NCC12, most preferably a Lactobacillus acidophilus (L. acidophilus) strain NCC12, as deposited under the Budapest treaty under the deposit numbers ATCC-4356 or DSM-20079.

According to a second aspect, the invention provides a composition comprising an effective amount of a pre-conditioned probiotic Lactobacillus strain, and additionally an effective amount of a galacto-oligosaccharide (GOS), wherein the composition has been prepared preferably according to a method as defined herein, preferably according to the first aspect of the invention. The inventive composition may comprise the pre-conditioned probiotic Lactobacillus strain in an amount of between 10³ cfu to 10¹² cfu, typically in an amount of between 10⁴ cfu to 10¹¹ cfu per daily dose, preferably in an amount of between 10⁵ cfu to 10¹⁰ cfu per daily dose, or 10⁵ cfu to 10⁹ cfu per daily dose, likewise preferably in an amount of between 10⁶ cfu to 10⁹ cfu per daily dose, 10⁶ cfu to 10⁸ cfu per daily dose or in an amount of 10⁸ cfu to 10¹⁰ cfu per daily dose, more preferably around 10⁷ cfu to 10⁹ cfu per daily dose. A preferred daily dose is around 10⁸ total cfu's per daily dose, e.g., 10⁷ to 10⁹ cfu per daily dose or 10⁸ to 10⁹ cfu per daily dose. The composition furthermore may comprise GOS in an amount of at least 1 or 2 g per daily dose, preferably at least 3 g per daily dose, likewise preferably between 2 to 12 g per daily dose, or between 2 to 10 g per daily dose, between 2 to 8 g per daily dose, or between 2 to 7 g per daily dose, more preferably between 3 to 12 g per daily dose, likewise more preferably between 3 and 10 g per daily dose, such as between 3 to 8 g per daily dose, between 3 to 7 g per daily dose, between 3 to 6 g per daily dose, or about 2, 3, 4, 5, 6, 7, 8, 9, 10, 11, or 12 g per daily dose or any range formed thereby.

As outlined herein, the inventive composition also allows or is for improving the probiotic effect, e.g., of an Lactobacillus strain in the intestinal tract of a subject, for promoting bacterial survival and/or metabolic activity and persistence in the intestinal tract of a subject, and/or for promoting a healthy microbiota in the intestinal tract of a subject. According to a particular embodiment, the improvement or increase of the probiotic effect is therefore a promotion of the survival or metabolic activity of a Lactobacillus strain, preferably as defined herein, in the colon and preferably a promotion of the health benefits associated to the lactobacillus strains, and/or is a promotion of a healthy microbiota in the intestinal tract of a healthy subject, preferably an increase of the presence of bifidobacteria and/or lactobacilli in the microbiota of a subject.

The inventive composition may be furthermore a solid or liquid, and/or may be present in form of a powder, a tablet, a capsule, or may be in form of an oil formulation, an emulsion, an oil-in-water emulsion (o/w emulsion), or a water-in-oil emulsion (w/o emulsion). Additionally, without being limiting thereto, the inventive composition may be in an administrable form, preferably selected from the group consisting of nutritional compositions, pharmaceutical formulations, dietary supplements, functional food products, functional beverage products, a nutritional composition for infant or children such as an infant formula or follow-on formula, and combinations thereof, e.g., a dairy product, a milk-based product, a whey protein-based beverage.

Again, the Lactobacillus strain is preferably a Lactobacillus acidophilus (L. acidophilus) strain, more preferably Lactobacillus acidophilus (L. acidophilus) strain NCC12, most preferably a Lactobacillus acidophilus (L. acidophilus) strain NCC12, as deposited under the Budapest treaty under the deposit numbers ATCC-4356 or DSM-20079.

In a third aspect, the invention provides a non-therapeutic use of the inventive composition comprising an effective amount of a pre-conditioned probiotic Lactobacillus strain supplemented with an effective amount of GOS, the composition being prepared according to the invention or as described herein, for increasing or boosting the probiotic effect, e.g. of an Lactobacillus strain in the digestive tract of a healthy subject: Increase of the probiotic effect may be manifested e.g., in improving the Lactobacillus strain metabolic activity and/or Lactobacillus strain survival in the intestinal tract of a healthy subject, increasing lactic acid and/or acetic acid production in the intestinal tract of a subject, promoting bacterial survival and persistence, and/or promoting a healthy microbiota in a healthy subject.

The inventive composition for non-therapeutic use preferably increases the presence of bifidobacteria and/or increases lactobacilli presence in the microbiota or intestinal tract of the healthy subject, increases survival and/or engraftment and/or viability of an Lactobacillus strain in the intestinal tract of the healthy subject, and/or increases the lactic acid/acetic acid production in the microbiota or intestinal tract of the healthy subject. According to a particular embodiment the inventive composition for non-therapeutic use therefore preferably concerns improving or increasing the probiotic effect, such as a promotion of the survival or metabolic activity of the Lactobacillus strain in the colon and thus a promotion of the health benefits associated to the lactobacillus strains; and/or a promotion of a healthy microbiota in the intestinal tract of the healthy subject, preferably an increase of the presence of bifidobacteria and/or lactobacilli in the microbiota or intestinal tract of the healthy subject. Moreover, the healthy subject is typically selected from an athlete, a child, a toddler or an infant (term infant or premature infant), an adult, an elderly, a pregnant woman, a vegetarian, a lactating woman, a companion animal, a cat, or a dog. A healthy subject in this context does usually not suffer from any disease. The inventive composition may be solid or liquid, and/or is present in form of a powder, a tablet, a capsule, or may be in form of an oil formulation, an emulsion, an oil-in-water emulsion (o/w emulsion), or a water-in oil emulsion (w/o emulsion). Moreover, the inventive composition may be in an administrable form, preferably selected from the group consisting of nutritional compositions, pharmaceutical formulations, dietary supplements, functional food products, functional beverage products, a nutritional composition for infants or children and combinations thereof, e.g., a dairy product, a milk-based product, a whey protein-based beverage. As before, the Lactobacillus strain is preferably a Lactobacillus acidophilus (L. acidophilus) strain, more preferably Lactobacillus acidophilus (L. acidophilus) strain NCC12, most preferably a Lactobacillus acidophilus (L. acidophilus) strain NCC 12, as deposited under the Budapest treaty under the deposit numbers ATCC-4356 or DSM-20079.

According to a fourth aspect, the invention provides a composition comprising an effective amount of a pre-conditioned probiotic Lactobacillus strain and an effective amount of GOS, preferably as prepared according to the invention or as described herein, for use as a medicament.

According to a fifth aspect, the invention provides a composition comprising an effective amount of a pre-conditioned probiotic Lactobacillus strain and an effective amount of GOS, preferably as prepared according to the invention or as defined herein, for use in the prevention and/or treatment of a disease in a patient in need thereof.

In the abovementioned first and second medical uses (fourth and fifth aspect) the patient is preferably at risk of suffering from a digestive dysfunction, an impaired microbiota, and/or has such a digestive dysfunction, an impaired microbiota or a condition requiring promotion of the development and/or growth of the microbiota.

The pre-conditioned probiotic Lactobacillus strain and additionally GOS typically increases the probiotic effect of a probiotic Lactobacillus strain, manifested e.g.; by a promotion of a healthy microbiota in the intestinal of tract of the patient, an increase of the presence of lactobacilli in the microbiota of the patient, and/or an increase of bacterial survival and persistence in the intestinal tract of a subject, preferably survival and persistence of the probiotic Lactobacillus strain in the intestinal tract of a subject. Additionally or alternatively, the promoted probiotic effect can encompass the promotion of the digestive functions, of the immune function and/or of the growth and development, especially in subjects suffered from impairment of their microbiota, and/or having a condition requiring such promotion. Such conditions may include e.g., any treatment or disease that diminishes the amount and/or function of (beneficial) bacteria in the microbiota, such as immunotherapies, cancer therapies, administration of antibiotics, patients suffering from diarrhea, from infections or inflammations, but may also and particularly concern any such and further conditions related to an impaired microbiota or a condition requiring promotion of the development and/or growth of the microbiota that affect infants and small children.

The inventive compositions applied for such uses may be solid or liquid, and/or may be present in form of a powder, a tablet, a capsule, or may be in form of an oil formulation, an emulsion, an oil-in-water emulsion (o/w emulsion), or a water-in oil emulsion (w/o emulsion). The inventive composition for such uses may also be in an administrable form, preferably selected from the group consisting of nutritional compositions, pharmaceutical formulations, dietary supplements, functional food products, functional beverage products, and combinations thereof, e.g., a dairy product, a milk-based product, a whey protein-based beverage. Particularly preferably, the probiotic Lactobacillus strain is a Lactobacillus acidophilus (L. acidophilus) strain, more preferably Lactobacillus acidophilus (L. acidophilus) strain NCC12, most preferably a Lactobacillus acidophilus (L. acidophilus) strain NCC12, as deposited under the Budapest treaty under the deposit numbers ATCC-4356 or DSM-20079.

Further preferred embodiments are described herein below and in the dependent claims.

### BRIEF DESCRIPTION OF THE DRAWINGS

FIG. 1: shows the short SHIME colonic model

FIG. 2: shows that survival of L. reuteri in presence or absence of GOS in the colon after 48 h incubation, particularly a positive effect of GOS on L. reuteri survival and an improved survival of pre-conditioned L. reuteri in presence of GOS compared to non preconditioned L. reuteri.

FIG. 3A: shows the LacS sequence of L. reuteri DSM 17938 (SEQ ID NO: 1)

FIG. 3B: shows the LacA sequences of L. reuteri DSM 17938 (SEQ ID NO: 2)

FIG. 4: shows the beta-galactosidase activity in L. acidophilus NCC12 and S. thermophilus NCC2496 and their pre-conditioned counterparts. Both preconditioned L. acidophilus NCC12 and preconditioned S. thermophilus NCC2496 strains exhibit a strong beta-galactosidase activity, shown by a constant conversion of X-gal substrate over the 2 h incubation, compared to their control strains grown on dextrose.

FIG. 5: shows the viability of pre-conditioned and non pre-conditioned L. acidophilus NCC12 in the colon in presence and absence of GOS. GOS promotes the survival of L. acidophilus NCC12 regardless the source of carbon to grow the strain. Both preconditioned (GOS grown) and non pre-conditioned (dextrose grown) L. acidophilus NCC12 survive better in the colon in presence of GOS. Preconditioned L. acidophilus NCC12 survives better in the colon in presence of GOS than its counterpart grown on dextrose, showing the positive effect of the preconditioning step on the survival of L. acidophilus NCC12 in the colon in presence of GOS.

FIG. 6: shows the survival of L. reuteri DSM17938, L. acidophilus NCC12 and Streptococcus thermophilus NCC2496 in presence or absence of GOS in the colon after 48 h incubation, particularly a positive effect of GOS on L. reuteri and L. acidophilus NCC12 survival and negative effect of GOS on Streptococcus thermophilus NCC2496 survival. While both L. acidophilus and S. thermophilus strains grow on GOS and exhibit strong beta-galactosidase activity (FIG. 4), only preconditioned L. acidophilus survives better in the colon in presence of GOS. S. thermophilus happens to miss Lac A in the genome. Pre-conditioned L. acidophilus survives well in the colon as also observed with pre-conditioned L. reuteri. Both L. reuteri and L. acidophilus have LacA gene, combined with high homology sequences for LacS (>70%). This means that presence of LacA gene combined with LacS having a sequence identity of at least 70% to the sequence found in L. reuteri DSM17938 is a feature that helps pre-conditioned strains surviving in the gut in presence of GOS.

### DETAILED DESCRIPTION

The present invention aims to provide solutions, compositions and uses which increase and promote a healthy microbiota and/or increase the survival or metabolic activity of certain bacteria in the intestinal tract. In detail, such solutions are compositions comprising galacto-oligosaccharide (GOS) pre-conditioned Lactobacillus strains in combination with GOS in the final product. The microbiota effect takes place in the gastrointestinal tract, such as in the duodenum, the jejunum, the ileum and/or in the colon.

The present invention also aims to provide methods for preparing a pre-conditioned probiotic Lactobacillus strain in combination with GOS in the final product such that an improved microbiota and/or survival or metabolic activity of probiotics in the gastrointestinal tract can be achieved by virtue of the combination of such pre-conditioned Lactobacillus strains in combination with GOS in the composition.

The invention herein provides a way of enhancing probiotic effects of Lactobacillus strains in a mammal, using specific substrate components during fermentation when manufacturing the probiotic Lactobacillus strains and further increasing the synbiotic effect between the probiotic Lactobacillus strain and GOS added in the final composition. The inventors of the present invention have developed a method which comprises the use of galacto-oligosaccharides (GOS) during cultivation of the probiotic Lactobacillus strain, so called pre-conditioning with GOS, which surprisingly results in an increased probiotic effect of the Lactobacillus strain, particularly an improved microbiota and/or survival or metabolic activity of probiotics within the gastrointestinal tract of a subject or patient upon administering the pre-conditioned probiotic Lactobacillus strain, as described herein. This effect is further boosted in the upon combined administration of GOS as a prebiotic and the pre-conditioned probiotic Lactobacillus strain to a subject or patient in need thereof. The main objective of the invention therefore relates to a method for preparing such a pre-conditioned probiotic Lactobacillus strain with GOS, to the non-therapeutical and therapeutical use of a pre-conditioned probiotic Lactobacillus strain in combination with GOS to improve microbiota and/or survival or metabolic activity of probiotics in the gastrointestinal tract of a mammal.

The improvement in microbiota and/or survival or metabolic activity of probiotics makes it possible to better dose the amounts of probiotics, to improve the efficacy of probiotics and also to decrease the dosage and/or the frequency of administration of the probiotic and further prebiotics, such as GOS, required to obtain the sought after health effects.

In view of the above, the following is disclosed.

### Methods for Pre-Conditioning Probiotic Lactobacillus Strain

According to the first aspect the invention provides a method for preparing a composition comprising a pre-conditioned probiotic Lactobacillus strain and GOS, characterized in that the method comprises the steps:
a) preconditioning of a probiotic Lactobacillus strain following the steps:
   i) cultivating a probiotic Lactobacillus strain in the presence of galacto-oligosaccharide (GOS) in a growth medium, thereby preconditioning the probiotic Lactobacillus strain; and
   ii) optionally harvesting the pre-conditioned probiotic Lactobacillus strain from the growth medium;
b) preparing a composition comprising the pre-conditioned probiotic Lactobacillus strain obtained from step a)i) or a)ii) and adding GOS to the composition,
wherein the probiotic Lactobacillus strain is provided from at least one Lactobacillus strain comprising a LacS transporter sequence having a sequence identity of at least 70% to SEQ ID NO: 1, and/or comprising a GH42 B-gal sequence, wherein the Lactobacillus strain is not a Lactobacillus reuteri strain.

In the context of the current invention the term “pre-conditioning” of the probiotic Lactobacillus strains preferably means growing, such as cultivating or fermenting, the probiotic Lactobacillus strains with GOS during the process of producing the probiotic Lactobacillus strains.

The inventive method therefore preferably comprises as a first step a)i) cultivating a probiotic Lactobacillus strain in the presence of GOS, thereby pre-conditioning the Lactobacillus strain. In the inventive context, the terms “cultivation” and “fermentation” are used interchangeably.

In optional step a)ii) the pre-conditioned probiotic Lactobacillus strain is then harvested from the growth medium. The term “pre-conditioned Lactobacillus strain” refers to a probiotic Lactobacillus strain produced by the inventive method including a pre-conditioning step with GOS. Harvesting of such a GOS pre-conditioned probiotic Lactobacillus strain is optional, as the GOS pre-conditioned probiotic Lactobacillus strains may also be used directly in step b) for preparing a composition comprising the pre-conditioned probiotic Lactobacillus strain obtained from step a)i) and adding GOS to the composition.

Furthermore, in step b) of the inventive method, the composition is formed by combining the pre-conditioned probiotic Lactobacillus strain obtained from step a) and further adding GOS to the final composition.

A Lactobacillus strain as used throughout the current invention may generally be any Lactobacillus strain, comprising a LacS transporter sequence having a sequence identity of at least 70% to SEQ ID NO: 1, and/or comprising a GH42 B-gal sequence, wherein the Lactobacillus strain is not a Lactobacillus reuteri strain. Lactobacillus reuteri is also known as Limosilactobacillus reuteri. A Lactobacillus strain as used throughout the current invention is typically publicly and/or commercially available.

Lactobacilli strains as defined above preferably include or concern all Lactobacilli strains as falling under the taxonomy “Lactobacillus” as applied before Wednesday 15 Apr. 2020 (Ex-Lactobacilli strains) and also all those Ex-Lactobacilli strains and Lactobacillus strains as reclassified in the International Journal of Systematic and Evolutionary Microbiology (IJSEM), released Wednesday 15 Apr. 2020, provided the Lactobacillus strain is not a Lactobacillus reuteri strain or, according to the new taxonomy a Limosilactobacillus reuteri strain. This reclassification classified former Lactobacilli strains (Ex-Lactobacilli strains) scattering the species of the Lactobacillaceae family under Lactobacillus, paralactobacillus, Pediococcus and 23 novel genera (Zheng J., Wittouck S., Salvetti E. et al., (2020). A taxonomic note on the genus Lactobacillus: Description of 23 novel genera, emended description of the genus Lactobacillus Beijerink 1901, and union of Lactobacillaceae and Leuconostocaceae. https://doi.org/10.1099/ijsem.0.004107), based on several genetic approaches and markers (average nucleotide identity, average aminoacid identity, core-gene aminoacid identity, core genome phylogeny, signature genes and metabolic or ecologic criteria).

Lactobacilli strains according to the current invention therefore include at least: Lactobacillus delbrueckii subsp. delbrueckii, Lactobacillus delbrueckii subsp. bulgaricus, Lactobacillus acidophilus, Lactobacillus crispatus, Lactobacillus gasser, Lactobacillus helveticus, Lactobacillus iners, Lactobacillus jensenii, and Lactobacillus johnsonii, which remain in the Lactobacillus genus, Lactobacillus casei, Lactobacillus paracasei, and Lactobacillus rhamnosus, which are termed according to the new taxonomy Lacticaseibacillus casei, Lacticaseibacillus paracasei, and Lacticaseibacillus rhamnosus,
Lactobacillus salivarius, now termed Ligilactobacillus salivarius,
Lactobacillus fermentum, now termed Limosilactobacillus fermentum,
Lactobacillus brevis, now termed 0, and
Lactobacillus kefiri, now termed Lentilactobacillus kefiri, etc.

In a preferred embodiment of the current invention, the Lactobacillus strain as used herein is provided from at least one Lactobacillus strain, comprising a LacS transporter sequence having a sequence identity of at least 70% to SEQ ID NO: 1, and/or comprising a GH42 B-gal sequence, wherein the Lactobacillus strain is not a Lactobacillus reuteri strain.

A GH42 B-gal sequence is generally known to a skilled person, and may be preferably selected from a GH42 B-gal sequence occurring in Lactobacilli, more preferably a GH42 B-gal sequence selected from a sequence having a sequence identity of at least 70% to SEQ ID NO: 2. Accordingly, in a preferred embodiment of the current invention, the Lactobacillus strain as used herein is provided from at least one Lactobacillus strain, comprising a LacS transporter sequence having a sequence identity of at least 70% to SEQ ID NO: 1, and/or comprising a GH42 B-gal sequence, having a sequence identity of at least 70% to SEQ ID NO: 2, again provided that the Lactobacillus strain is not a Lactobacillus reuteri strain.

Particularly preferably, the probiotic Lactobacillus strain is a Lactobacillus acidophilus (L. acidophilus) strain as known to a skilled person, more preferable Lactobacillus acidophilus (L. acidophilus) strain NCC12, most preferably a Lactobacillus acidophilus (L. acidophilus) strain NCC12, as deposited under the Budapest treaty under the deposit numbers ATCC-4356 or DSM-20079.

The term “sequence identity” as defined herein means that the sequences are compared as follows. To determine the percent identity of two amino acid sequences, the sequences can be aligned for optimal comparison purposes (e.g., gaps can be introduced in the sequence of a first amino acid sequence). The amino acids at corresponding amino acid positions can then be compared. When a position in the first sequence is occupied by the same amino acid as the corresponding position in the second sequence, then the molecules are identical at that position. The percent identity between the two sequences is a function of the number of identical positions shared by the sequences. E.g. where a particular peptide is said to have a specific percent identity to a reference polypeptide of a defined length, the percent identity is relative to the reference peptide. Thus, a peptide that is 50% identical to a reference polypeptide that is 100 amino acids long can be a 50 amino acid polypeptide that is completely identical to a 50 amino acid long portion of the reference polypeptide. It might also be a 100 amino acid long polypeptide, which is 50% identical to the reference polypeptide over its entire length. Of course, other polypeptides will meet the same criteria. Such a determination of percent identity of two sequences can be accomplished using a mathematical algorithm. A preferred, non-limiting example of a mathematical algorithm utilized for the comparison of two sequences is the algorithm of Karlin et al. (1993), PNAS USA, 90:5873-5877. Such an algorithm is incorporated into the NBLAST program, which can be used to identify sequences having the desired identity to the amino acid sequence of the invention. To obtain gapped alignments for comparison purposes, Gapped BLAST can be utilized as described in Altschul et al. (1997), Nucleic Acids Res, 25:3389-3402. When utilizing BLAST and Gapped BLAST programs, the default parameters of the respective programs (e.g., NBLAST) can be used. The sequences further may be aligned using Version 9 of the Genetic Computing Group's GAP (global alignment program), using the default (BLOSUM62) matrix (values −4 to +11) with a gap open penalty of −12 (for the first null of a gap) and a gap extension penalty of −4 (per each additional consecutive null in the gap). After alignment, percentage identity is calculated by expressing the number of matches as a percentage of the number of amino acids in the claimed sequence. The described methods of determination of the percent identity of two amino acid sequences can be applied correspondingly to nucleic acid sequences.

The term “GOS” as used herein means “Galacto-oligosaccharide”. Galacto-oligosaccharides (GOS) as used herein typically consist of β-linked galactose moieties with galactose or glucose at the reducing end. Such GOS contain β-(1→2), β-(1→3), β-(1→4), or β-(1→6) linked galactose moieties and may have a degree of polymerization (DP) of 3-8 galactose units. The term GOS is therefore preferably referred to as oligosaccharide(s) comprising at least three galactose units, more preferably as oligosaccharide(s) comprising at least four galactose units, In one embodiment having a degree of polymerization (DP) of 3-8 or 4-8 galactose units. Oligosaccharides occur naturally in the milk of some mammals, e.g., cow and human. GOS are commercially available and can be synthesized via biosynthesis processes from lactose by trans-galactosidase activity of β-galactosidases. GOS formation in biosynthesis procedures is usually favored by high concentrations of lactose or lactulose, incomplete lactose turnover, low water activity, and the use of enzymes with preference for trans-galactosylation. The linkage type(s) of resulting GOS from such processes is(are) specific for the enzymes used for biosynthesis.

One particular type of mixture of carbohydrates that can advantageously be used herein as a source of GOS to grow the probiotic bacteria is a mixture of GOS and of cow's milk oligosaccharides. In particular mixtures of GOS with 3′-sialyllactose (3′-SL or 3SL) and/or 6′-sialyllactose (6′-SL or 6SL) are preferably used. Indeed, such mixtures contain some oligosaccharides similar to human milk, which is particularly advantageous when the composition of the invention is used as an infant formula or as a nutritional supplement for infants. Such advantageous effects are described for example in Simeoni et al.; “Gut microbiota analysis reveals a marked shift to bifidobacteria by a starter infant formula containing a synbiotic of bovine milk-derived oligosaccharides and Bifidobacterium animalis subsp. lactis CNCM 1-3446”; Environ Microbiol, 2016, 18(7): 2185-2195. Such compositions can typically be obtained from concentrating whey permeate to obtain a concentrated bovine milk oligosaccharide composition and either adding GOS or generating the GOS in situ from hydrolysis of lactose by the action of a 6-galactosidase.

The GOS source used in step a)i of the inventive method to pre-condition the probiotic Lactobacillus strain and the GOS source further added in step b) for preparing a final composition comprising the pre-conditioned probiotic Lactobacillus strain with GOS, can be provided in the form of essentially pure GOS (i.e. ingredient having at least 90% GOS) or as part of a mixture, such as a mixture of carbohydrates, comprising GOS.

The essential aspect for the pre-conditioning of a probiotic Lactobacillus strain with GOS is that a sufficient amount of GOS is provided in the growth medium. According to one embodiment the GOS source is therefore typically added to the growth medium in step a)i) of the method for preparing a pre-conditioned probiotic Lactobacillus strain in an amount such as providing at least 0.2 wt %, preferably at least 0.5 wt %, even more preferably at least 0.75 wt % of GOS in the growth medium. In a preferred embodiment, GOS is provided in an amount such as providing at most 8 wt % of GOS, such as about 7 wt % GOS, 6 wt % GOS, or even 5 wt % GOS at most, more preferably at most 4 wt % of GOS or even 3 wt % GOS, in the growth medium, preferably calculated on basis of the growth medium (% w/w). Any combination of these upper and lower ranges is encompassed herewith. Preferably, GOS is added to the growth medium in a range of 0.2 wt % to 8 wt % or even 0.2 wt % to 7 wt %, or 0.2 wt % to 6 wt %, more preferably in a range of 0.5 wt % to 8 wt % or even 0.5 wt % to 7 wt %, or 0.5 wt % to 6 wt %, even more preferably in a range of 0.75 wt % to 8 wt % or even 0.75 wt % to 7 wt %, or 0.75 wt % to 6 wt %, and most preferably in a range of 0.75 wt % to 7 wt %, in a range of 0.75 wt % to 6 wt % or even in a range of 0.75 wt % to 5 wt %, such as a range of 0.75 wt % to 5 wt % or 0.75 wt % to 4 wt %, preferably calculated on basis of the growth medium.

When a mixture of carbohydrates is used as a source of GOS for pre-conditioning a probiotic Lactobacillus strain or when preparing the final composition containing the pre-conditioned probiotic Lactobacillus strain with GOS, it is preferred that the amount of GOS in such a mixture is at least 20 wt %, preferably at least 30 wt %, more preferably at least 40 wt %, even more preferably at least 45 wt %, most preferably at least 48 wt %, preferably calculated on a dry weight basis of the mixture containing GOS.

Although it is not necessary that the bacteria consume only GOS as carbon source during the fermentation, high proportion of GOS in the GOS source during the pre-conditioning step favors the consumption of GOS by the bacteria during fermentation over consumption of other carbohydrates, leading to improved pre-conditioning effect. Smaller carbohydrates such as di-saccharides (lactose for instance) can also be present and be consumed by the bacteria supporting the pre-conditioning effect. Accordingly, it is also preferred that during the pre-conditioning step the GOS source comprises at most 55 wt %, preferably at most 50 wt %, more preferably at most 45 wt %, most preferably at most 42 wt % of mono- or di-saccharides and/or no more than 40 wt %, preferably no more than 30 wt % lactose, preferably calculated on a dry weight basis of the mixture containing GOS. As an example, a mixture of carbohydrate used as a source of GOS during the pre-conditioning step may comprise GOS in amounts defined above for the mixture and the remainder formed by lactose, glucose, and/or galactose, and optionally 3′SL and/or 6′-SL.

The culture medium, in an embodiment, also comprise an electron acceptor such as fructose, citrate, glycerol and/or 1,2 propanediol.

The culture medium, in an embodiment, also comprise fructose, such as acting as an electron acceptor. In illustrative, but non-limiting, examples the culture medium may comprise at least 0.5 wt % fructose, preferably at least 1 wt % fructose and more preferably at least 2 wt % fructose, preferably calculated on a dry weight basis of the culture medium. Correspondingly, in illustrative, but non-limiting, examples the culture medium preferably does not comprise more than 20 wt % fructose, preferably no more than 15 wt % fructose and more preferably no more than 10 wt % fructose.

According to a further embodiment, addition of GOS in step a) of the method for preparing a pre-conditioned probiotic Lactobacillus strain to the growth medium may occur at any point of time of the cultivation step a)i), e.g., at the start of cultivation of probiotic Lactobacillus strain, in the middle or at the end of cultivation of probiotic Lactobacillus strain in step a)i), wherein addition may occur either at once, stepwise or continuously.

The cultivation step a)i) is carried out in a way that is well known to the person skilled in the art. Cultivation in step a)i) includes the steps of inoculation of sterile standard growth medium with a defined amount of bacteria (cfu or afu), followed by incubation under defined temperature (usually 37° C.) and pH. In this context, a defined amount of bacteria (cfu or afu) for starting an inoculation of sterile standard growth medium may be determined by a skilled person according to common general knowledge and practice in the art. Suitable yields can be obtained with the growth medium comprising GOS as described above, preferably without changing the cultivation conditions compared to what the person skilled in the art would use for cultivation of the same strain with a standard growth medium.

A standard growth medium for cultivating/fermenting the Lactobacillus strain in step a)i) of the current invention may be any known standard growth medium used for lactic acid bacteria (LAB), such as MRS medium, or any further suitable medium. Such a standard growth medium is preferably commercially available but may be also produced by addition of compounds according to well-known standard recipes. A standard growth medium for the Lactobacillus strain may typically contain carbohydrates, such as simple sugars selected e.g., from dextrose, sucrose, maltose, fructose or lactose, various nitrogen sources, such as peptone, yeast extract, beef extract, or whey protein, minerals, mainly Mn²⁺ and Mg²⁺, and buffering agents, such as sodium acetate (CH₃COONa), trisodium citrate (Na₃C₆H₅O₇), or Di-sodium-glycerophosphate (C₃H₇Na₂O₆ P) are commonly used buffers in LAB media. Other components that have been used in standard growth media with buffering activity may include disodium phosphate (Na₂HPO₄), ammonium citrate (NH₄C₆H₅O₇), trisodium phosphate (Na₃PO₄), potassium biphosphate (KH₂PO₄), magnesium phosphate tribasic Mg₃(PO₄)₂, calcium carbonate (CaCO₃), and dipotassium phosphate (K₂HPO₄). Such standard growth media may also contain a wide range of growth factors, surfactants, such as lecithin or Tweens (e.g., Tween 20, 80 and 85). The cultivation/fermentation may be carried out under anaerobic or aerobic conditions, depending on the strain to be produced, but preferably under anaerobic conditions. Also, the pH may be controlled or not, depending on the conditions known to be the best for a specific strain to grow. The temperature and duration of the cultivation step is variable from one strain to another and is also well-known to the person skilled in the art of probiotic the Lactobacillus strain cultivation.

According to one embodiment the cultivation/fermentation of the probiotic Lactobacillus strain in step a)i) of the inventive method occurs over a considerable period of time to allow a pre-conditioning of the probiotic Lactobacillus strain in the presence of GOS, typically for a period of at least 1 hour, preferably for a period of between 5 to 18 hours, likewise preferably 7 to 14 hours, more preferably for a period of between 10 to 13 hours, and even more preferably for a period of about 12 hours.

Moreover, according to a further embodiment the cultivation/fermentation of the probiotic Lactobacillus strain in step a)i) of the inventive method occurs at a temperature of between 25 to 45° C., preferably between 30 and 40° C., and even more preferably at a temperature of between 35 and 39° C., such as about 37° C.

Additionally, in another embodiment the cultivation/fermentation of the probiotic Lactobacillus strain in step a)i) of the inventive method occurs at a pH of between 3.0 and 7. The pH during pre-conditioning of probiotic Lactobacillus strain during cultivation step a) may be either not controlled or set to a constant value throughout the cultivation step a)i), typically to be monitored and adjusted during cultivation step a). Accordingly, the pH may be between 3.0 and 7, particularly when pH is not controlled during pre-conditioning of probiotic Lactobacillus strain. Alternatively, if pH is controlled during cultivation step a)i), pH may be adjusted to a range of about 5.0 to 7.0, e.g., about 5.0 to 6.0 or about 6.0 to 7.0, preferably pH is adjusted to a range of about 6.0 to 7.0, more preferably to a range of about 6.6±0.4, most preferably to a range of about 6.6±0.2.

After cultivation/fermentation of a probiotic Lactobacillus strain in step a)i) of the inventive method the thereby pre-conditioned probiotic Lactobacillus strain is harvested in step a)ii). The harvesting step, which aims at separating the bacterial cells from the growth medium, is also carried out in a way that is well known to the person skilled in the art, such as by concentrating the bacteria, centrifugation, filtration, membrane-filtration, decantation, etc., preferably centrifugating or concentrating the probiotic Lactobacillus strain. Typically, harvesting of the pre-conditioned probiotic Lactobacillus strain occurs in step a)ii) after a period of at least 1 hour as defined above for cultivation/fermentation in step a)i).

After harvesting the pre-conditioned probiotic Lactobacillus strain in step a)ii) the harvested cells may be washed in an optional step a)iii) prior to further processing, typically to remove traces of growth medium (after cultivation/fermentation). Washing may occur with either saline water, brine, or any further suitable liquid.

Either directly after harvesting the pre-conditioned probiotic Lactobacillus strain from the growth medium according to step a)ii) or after an optional washing step a)iii) the pre-conditioned probiotic Lactobacillus strain may be subjected to a drying step a)iv). Such a drying step a)iv) may be carried out by any method known to a skilled person, such as spray-drying, fluid bed drying, air convective drying, atmospheric drying, roller drying or freeze drying, lyophilizing, and more preferably spray-drying or freeze drying. Spray drying may also be carried out using in the presence of protecting agents, e.g., as described in WO 2017/001590.

Optionally, the pre-conditioned probiotic Lactobacillus strain obtained after harvesting from the growth medium and/or after washing the pre-conditioned probiotic Lactobacillus strain may further be mixed with protective agents, such as protectants, cryoprotectants and/or carriers before the drying step, as known to the person skilled in the art and as appropriate depending on the drying method to be used and the bacteria to be dried. Such protectants, cryoprotectants and/or carriers typically include glycerol, skimmed milk, serum albumin, peptone, yeast extract, saccharose, glucose, sorbitol, malt extract, trehalose etc. Commercially available protectants or cryoprotectants include e.g., ‘Unipectine™ RS 150, etc. or are as described in EP 14 744 005.1, US 2014/0004083 A1, US 2016/0298077 A1, etc.

Alternatively, or additionally, the pre-conditioned probiotic Lactobacillus strain obtained after culturing, after harvesting from the growth medium or after washing or even after drying the pre-conditioned probiotic Lactobacillus strain, i.e. obtained after any of steps a)i, a)ii) or a)iii) after a)iv) may be encapsulated for further preservation and/or use, e.g. by formation of microspheres containing the pre-conditioned probiotic Lactobacillus strain using common encapsulation agents, e.g. alginate, alginate/pullulan, starch, xanthan, xanthan-gellan gum mixtures, gelatin, cellulose acetate phethalate, chitosan, or any further suitable encapsulation material. Encapsulation is well known and can be carried out by a person skilled in the art.

Furthermore, the inventive method for preparing a composition comprising a pre-conditioned probiotic Lactobacillus strain and GOS, comprises the step b) of preparing a composition comprising the pre-conditioned probiotic Lactobacillus strain obtained from step a) and adding GOS to the composition. GOS may be applied in amounts as defined above, either pure or as a mixture as defined before.

According to a preferred embodiment, GOS may be added in step b) of the inventive method to the pre-conditioned probiotic Lactobacillus strain obtained from step a) in an amount of at least 1 or 2 g per daily dose, preferably at least 3 g per daily dose, likewise preferably between 2 to 12 g per daily dose, or between 2 to 10 g per daily dose, between 2 to 8 g per daily dose, or between 2 to 7 g per daily dose, more preferably between 3 to 12 g per daily dose, likewise more preferably between 3 and 10 g per daily dose, such as between 3 to 8 g per daily dose, between 3 to 7 g per daily dose, between 3 to 6 g per daily dose, or about 2, 3, 4, 5, 6, 7, 8, 9, 10, 11, or 12 g per daily dose or any range formed thereby, to prepare the (final) composition. The GOS added to the composition comprising the pre-conditioned probiotic Lactobacillus strain prepared in step b) can have a degree of polymerization (DP) of 3-8.

### Compositions Comprising the Pre-Conditioned Probiotic Lactobacillus Strain and Gos

According to the second aspect, the invention provides a composition comprising an effective amount of a pre-conditioned probiotic Lactobacillus strain and additionally an effective amount of a galacto-oligosaccharide (GOS), wherein the composition has been prepared/obtained according to the inventive method. Such a pre-conditioning of a probiotic Lactobacillus strain in the presence of GOS is typically carried out according to the inventive method as described herein under the first aspect of the invention and also as defined in the claims as attached. As already disclosed above in detail, cultivating/fermentation of a probiotic Lactobacillus strain in the presence of GOS preferably occurs over a time sufficient to pre-condition a probiotic Lactobacillus strain accordingly, e.g., over a period of at least one hour, preferably according to a process as defined herein.

Typically, an “effective amount” of a pre-conditioned probiotic Lactobacillus strain as defined herein for the composition may comprise a pre-conditioned probiotic Lactobacillus strain typically in an amount of between 10³ cfu to 10¹² cfu, typically in an amount of between 10⁴ cfu to 10¹¹ cfu per daily dose, preferably in an amount of between 10⁵ cfu to 10¹⁰ cfu per daily dose or 10⁵ cfu to 10⁹ cfu per daily dose, likewise preferably in an amount of between 10⁶ cfu to 10⁹ cfu per daily dose, 10⁶ cfu to 10⁸ cfu per daily dose or in an amount of 10⁸ cfu to 10¹⁰ cfu per daily dose. A preferred daily dose is around 10⁸ total cfu's per daily dose, e.g., 10⁷ to 10⁹ or 10⁸ to 10⁹ cfu per daily dose.

As before, the Lactobacillus strain is preferably any Lactobacillus strain known to a skilled person and preferably as defined above, comprising a LacS transporter sequence having a sequence identity of at least 70% to SEQ ID NO: 1, and/or comprising a GH42 B-gal sequence, wherein the Lactobacillus strain is not a Lactobacillus reuteri strain. Particularly preferably, the probiotic Lactobacillus strain is a Lactobacillus acidophilus (L. acidophilus) strain, more preferably Lactobacillus acidophilus (L. acidophilus) strain NCC12, most preferably a Lactobacillus acidophilus (L. acidophilus) strain NCC12, as deposited under the Budapest Treaty under the deposit numbers ATCC-4356 or DSM-20079. A GH42 B-gal sequence is generally known to a skilled person, and may be preferably selected from a GH42 B-gal sequence occurring in Lactobacilli, more preferably from a GH42 B-gal sequence having a sequence identity of at least 70% to SEQ ID NO: 2.

Moreover, an “effective amount” of a galacto-oligosaccharide (GOS) as defined herein for the composition may comprise GOS in an amount of at least 1 or 2 g per daily dose, preferably at least 3 g per daily dose, likewise preferably between 2 to 12 g per daily dose, or between 2 to 10 g per daily dose, between 2 to 8 g per daily dose, or between 2 to 7 g per daily dose, more preferably between 3 to 12 g per daily dose, likewise more preferably between 3 and 10 g per daily dose, such as between 3 to 8 g per daily dose, between 3 to 7 g per daily dose, between 3 to 6 g per daily dose, or about 2, 3, 4, 5, 6, 7, 8, 9, 10, 11, or 12 g per daily dose or any range formed thereby. The addition of GOS to the pre-conditioned probiotic Lactobacillus strain as defined herein provides further improved synbiotic properties to the final product.

Accordingly, the pre-conditioning of the probiotic Lactobacillus strain modifies the probiotic Lactobacillus strain so that the Lactobacillus strain have different and improved properties as compared to a non-pre-conditioned probiotic Lactobacillus strain, i.e., a probiotic Lactobacillus strain that does not have been pre-conditioned by cultivating or culturing in the presence of GOS. These different and improved properties include, among others, the above listed and in the properties disclosed in the examples.

The inventive composition comprising an effective amount of a pre-conditioned probiotic Lactobacillus strain and additionally an effective amount of a galacto-oligosaccharide (GOS), as defined herein may be any type of composition in which probiotic bacteria can be incorporated, such as a food product, a beverage, an animal feed product, a nutritional supplement for human or animal, a pharmaceutical composition or a cosmetic composition. More preferably, the inventive composition may be provided in an administrable form, preferably selected from the group consisting of food products, beverages, pharmaceutical formulations, dietary or nutritional supplements, functional food products, functional beverage products, nutraceuticals, and combinations thereof.

The inventive composition may be solid or liquid. Preferably it is present in form of a powder, a tablet, a capsule, or may be in form of an oil formulation, an emulsion, e.g., an oil-in-water emulsion (o/w emulsion), a water-in oil emulsion (w/o emulsion), etc. If present in powder form it can be intended to be used by the final consumer in solid (such as powder form) or semi-solid form (such as for example in the form of a paste) or, alternatively, to be reconstituted into a liquid before use.

Food products and beverages as defined herein may include all products intended to be consumed orally by human beings, for the purpose of providing nutrition and/or pleasure. The expression “food product” as well as the term “beverages” usually mean compositions which nourish a subject. This “food product” is usually to be taken orally or intraperitoneally, and it can include a lipid or fat source and a protein source. Likewise, a “beverage” is usually to be taken orally, is liquid or a semi-liquid, and can include a lipid or fat source and a protein source.

Food products and beverages as defined herein can for example include a nutritional composition, preferably for human consumption, such as for infants and/or young children, for a pregnant or lactating woman or a woman desiring to get pregnant, for individuals in need of a special nutrition due to an adverse health condition or for elderly people. More preferably, the nutritional composition is selected from infant formula, infant cereals, follow-up or follow-on formula, growing-up milks, functional milks, baby food, infant cereal compositions, and milk products for pregnant and lactating women or for women desiring to get pregnant. Other examples of food products and beverages include sweet and savory snacks, powdered drinks, cereal products and dairy products, such as milk products, whey protein-based products, etc. According to one particular preferred embodiment, the inventive composition is an infant formula, a follow-on formula, a growing-up milk or a product for pregnant or lactating women. In one further particular embodiment the inventive composition may be an infant formula.

The inventive composition can also be in the form of an animal food product or a nutritional supplement for animals. Preferably, the animal is a mammal. Examples of animals include, cows, sheep, goats, horses, dogs, cats, rabbits, rats, mice, fish, birds and the like.

The expression “infant formula” as used herein refers to a foodstuff intended for particular nutritional use by infants during the first months of life and satisfying by itself the nutritional requirements of this category of person (Article 2(c) of the European Commission Directive 91/321/EEC 2006/141/EC of 22 Dec. 2006 on infant formulae and follow-on formulae). It also refers to a nutritional composition intended for infants and as defined in Codex Alimentarius (Codex STAN 72-1981) and Infant Specialities (incl. Food for Special Medical Purpose). The expression “infant formula” encompasses both “starter infant formula” and “follow-up formula” or “follow-on formula”.

A “follow-up formula” or “follow-on formula” is an infant formula given from the 6th month onwards. It constitutes the principal liquid element in the progressively diversified diet of this category of person.

The expression “baby food” means a foodstuff intended for particular nutritional use by infants or young children during the first years of life.

In the inventive context, the term “infant” means a child under the age of 12 months. Moreover, the expression “young child” means a child aged between one and three years, also called toddler.

The expression “infant cereal composition” means a foodstuff intended for particular nutritional use by infants or young children during the first years of life.

The product can also be in the form of an animal food product or a nutritional supplement for animals. Preferably, the animal is a mammal. Examples of animals include, cows, sheep, goats, horses, dogs, cats, rabbits, rats, mice, fish, birds and the like.

Dietary or nutritional supplements are typically present in the form of a liquid, such as a refrigerated liquid, in form of a powder or a tablet or capsule, an oil formulation, an emulsion, e.g., an oil-in-water emulsion (o/w emulsion), a water-in oil emulsion (w/o emulsion), etc. as mentioned above. Preferably it is in the form of a powder, a tablet, a capsule or an oil formulation. Powder supplements typically encompass supplements to be dissolved in a liquid or to be sprinkled on food or in a beverage. Such supplements are intended to provide additional nutrients and/or a health benefit to the subject consuming it, as well as other beneficial ingredients, such as the herein-defined and prepared pre-conditioned probiotic bacteria Lactobacillus strain and additionally an effective amount of a galacto-oligosaccharide (GOS). A supplement according to the present invention can therefore be used for providing nutrients and/or a health benefit to human beings, as well as to animals, as defined above. Dietary or nutritional supplements include for example the herein-defined and prepared pre-conditioned probiotic Lactobacillus strain and additionally an effective amount of a galacto-oligosaccharide (GOS) as a powder supplement to be added to any sort of dietary or nutritional composition.

Pharmaceutical products include powder, tablet or capsule products intended to treat or prevent an adverse medical condition in a subject in need thereof, or to promote a favorable health condition.

Cosmetic compositions are typically intended for an aesthetic effect on the body and may be for topical use or may be administered by oral route, in the form of a powder, tablet or capsule.

### Applications of Inventive Compositions Comprising Pre-Conditioned Probiotic Lactobacillus Strain and Gos

The present invention aims to provide solutions, compositions and uses, which increase and promote a healthy microbiota and/or increase the survival or metabolic activity of certain bacteria in the intestinal tract. In detail, such solutions are compositions as described herein comprising an effective amount of galacto-oligosaccharides (GOS) pre-conditioned probiotic Lactobacillus strains and additionally an effective amount of a galacto-oligosaccharide (GOS) to be administered to a subject or a patient in need thereof, e.g., a patient being at risk of suffering from a digestive dysfunction, an impaired microbiota, and/or who has such a digestive dysfunction, an impaired microbiota or a condition requiring promotion of the development and/or growth of the microbiota. The microbiota effect takes place in the gastrointestinal tract, such as in the duodenum (first part of small intestine), the jejunum (middle part of small intestine) and/or in the colon (large intestine).

As shown herein inventive compositions allow for enhancing probiotic effects of Lactobacillus strains in a mammal, using GOS as specific substrate components during fermentation when manufacturing the probiotic Lactobacillus strains and supplementing such pre-conditioned probiotic Lactobacillus strain even further with galacto-oligosaccharides (GOS). There herein disclosed method specifically pre-conditions the probiotic Lactobacillus strain by use of galacto-oligosaccharides (GOS) during cultivation of the probiotic Lactobacillus strain, such that—further to the addition of GOS—an increased probiotic effect of the Lactobacillus strain occurs in vivo, particularly during non-therapeutic and therapeutic administrations as disclosed herein.

An increased metabolic activity of the probiotic Lactobacillus strain supplemented with galacto-oligosaccharides (GOS) is observed and can become evident e.g., through an increased lactic acid production upon administering the pre-conditioned probiotic Lactobacillus strain supplemented with galacto-oligosaccharides (GOS), as described herein. Such changes also indicate that the population of beneficial bacteria, such as bifidobacteria and other lactobacilli, can be increased and/or the microbiota and/or metabolic activity increased. The improvement in microbiota and/or survival or metabolic activity of probiotics of Lactobacillus strain obtained by the pre-conditioning step and supplemented with galacto-oligosaccharides (GOS), conferred to a subject and/or patient in need thereof makes it possible to decrease the dosage and/or the frequency of administration of the probiotic required to obtain the sought after health effects.

In the following, without being limiting thereto, exemplary therapeutic and non-therapeutic applications (uses) are listed.
NON-THERAPEUTIC USES OF COMPOSITIONS COMPRISING PRE-CONDITIONED PROBIOTIC LACTOBACILLUS STRAIN

According to the third aspect, the invention provides a non-therapeutic use of a composition comprising an effective amount of a pre-conditioned probiotic Lactobacillus strain and an effective amount of galacto-oligosaccharides (GOS) prepared according to the invention or as described herein, typically in form of an administrable composition, to increase or boost the probiotic effect of said Lactobacillus strain in the digestive tract of a healthy subject. Such an increase or boosting of the probiotic effect by a pre-conditioned probiotic Lactobacillus strain supplemented with galacto-oligosaccharides (GOS) in the digestive tract of a healthy subject typically can be determined in comparison with the not yet pre-conditioned probiotic Lactobacillus strain in the digestive tract of a healthy subject with or without supplementation with galacto-oligosaccharides (GOS).

The increase or boosting of the probiotic effect, typically of said probiotic Lactobacillus strain in the digestive tract of a healthy subject, inter alia but not exclusively, concerns the increase and promotion of a healthy microbiota and/or increase of the survival or increase of metabolic activity of certain bacteria in the intestinal tract. Increase of the probiotic effect may be manifested e.g., in improving the Lactobacillus strain metabolic activity and/or Lactobacillus strain survival in the intestinal tract of a healthy subject, promoting bacterial survival and persistence, promoting a healthy microbiota in a healthy subject.

In this context, a healthy subject, which is the main addressee of this non-therapeutic use, may be selected from an athlete, a child, a toddler or an infant, an adult, an elderly, a pregnant woman, a vegetarian, a lactating woman, a companion animal, a cat, or a dog, preferably for addressing or even improving such body functions and body processes. Such a healthy subject usually does not suffer from any condition or disease e.g., as described herein, such as digestive dysfunctions, an impaired microbiota, and/or a digestive dysfunction.

In these subjects the effect of the non-therapeutic use can be promotion of the immune functions, balance or rebalance the microbiota, promotion of the intestinal function, or general growth or development.

Any of the methods as discussed before for pre-conditioning a probiotic Lactobacillus strain as well as any of the features of compositions as defined herein comprising the pre-conditioned probiotic Lactobacillus strain supplemented with galacto-oligosaccharides (GOS) apply mutatis mutandis for the third aspect of the current invention, namely to the non-therapeutic use of the inventive compositions as described above.

This specifically concerns features of the method of pre-conditioning a probiotic Lactobacillus strain, the preparation of the composition with the pre-conditioned probiotic Lactobacillus strain supplemented with galacto-oligosaccharides (GOS) and any ingredients and amounts and further features as used for compositions comprising the pre-conditioned probiotic Lactobacillus strain. The composition may be provided in any form as depicted above.

In this context, it is e.g., noted that the inventive composition for non-therapeutic purposes may be solid or liquid, and/or is present in form of a powder, a tablet, a capsule, or may be in form of an oil formulation, an emulsion, an oil-in-water emulsion (o/w emulsion), or a water-in oil emulsion (w/o emulsion).

Moreover, the inventive composition for non-therapeutic purposes may be in an administrable form, preferably selected from the group consisting of nutritional compositions, pharmaceutical formulations, dietary supplements, functional food products, functional beverage products, nutritional composition for infants or children, such as infant formula or follow-on formula, and combinations thereof, e.g., a dairy product, a milk-based product, a whey protein-based beverage.

As before, the Lactobacillus strain is particularly preferable any Lactobacillus strain known to a skilled person and preferably as defined above, comprising a LacS transporter sequence having a sequence identity of at least 70% to SEQ ID NO: 1, and/or comprising a GH42 B-gal sequence, wherein the Lactobacillus strain is not a Lactobacillus reuteri strain. Particularly preferably, the probiotic Lactobacillus strain is a Lactobacillus acidophilus (L. acidophilus) strain, more preferably Lactobacillus acidophilus (L. acidophilus) strain NCC12, most preferably a Lactobacillus acidophilus (L. acidophilus) strain NCC12, as deposited under the Budapest treaty under the deposit numbers ATCC-4356 or DSM-20079.
USE OF COMPOSITIONS COMPRISING PRE-CONDITIONED PROBIOTIC LACTOBACILLUS STRAIN AND GOS AS A MEDICAMENT

According to the fourth aspect, the invention also provides a composition comprising an effective amount of a pre-conditioned probiotic Lactobacillus strain supplemented with galacto-oligosaccharides (GOS) as prepared according to the invention or as described herein for use as a medicament (first medical use).

Similar as before, the effect of such a composition as a medicament is particularly related to the increase of the synbiotic pre- and probiotic effects in the digestive tract of a patient in need of such a medicament, usually compared to the effect of a probiotic Lactobacillus strain not conditioned with GOS or usual combinations of pre- and probiotics.

The therapeutic effects that are related to the use of the inventive compositions as a medicament inter alia address effects related to any of the herein mentioned diseases and applications, particularly in patients that are at risk or that are already suffering from any of such diseases, particularly diseases as mentioned explicitly herein in the context of therapeutic applications (see also below, second medical uses). Such patients may be e.g., at risk of suffering from a digestive dysfunction, an impaired microbiota, and/or has a digestive dysfunction, an impaired microbiota or a condition requiring promotion of the development and/or growth of the microbiota. The medicament may also be for use in improving or increasing the probiotic effect, preferably of the probiotic Lactobacillus strain in the gastrointestinal tract of the patient. Such an improving or increasing the probiotic effect is e.g. a promotion of a healthy microbiota in the intestinal tract of the patient, preferably an increase of the presence of bifidobacteria and/or lactobacilli in the microbiota of the patient; and/or is a promotion of the digestive functions and/or the immune functions and/or growth.

Any of the methods as discussed before for pre-conditioning a probiotic Lactobacillus strain as well as any of the features of compositions as defined herein comprising the pre-conditioned probiotic Lactobacillus strain supplemented with galacto-oligosaccharides (GOS) also apply mutatis mutandis for the fourth aspect of the current invention, namely to the use of the inventive compositions as a medicament.

This specifically concerns features of the method of preparing a composition comprising a pre-conditioned probiotic Lactobacillus strain supplemented with galacto-oligosaccharides (GOS), and any ingredients and amounts and further features as used for the inventive compositions in any form as depicted above.

In this context, it is particularly preferred that the inventive composition for use as a medicament may be solid or liquid, may be present in form of a powder, a tablet, a capsule, or may be in form of an oil formulation, an emulsion, an oil-in-water emulsion (o/w emulsion), or a water-in oil emulsion (w/o emulsion).

Likewise, the inventive composition for use as a medicament may be in an administrable form, preferably selected from the group consisting of nutritional compositions, pharmaceutical formulations, dietary supplements, functional food products, functional beverage products, and combinations thereof, e.g., a dairy product, a milk-based product, a whey protein-based beverage.

As before, the Lactobacillus strain is particularly preferable any Lactobacillus strain known to a skilled person and preferably as defined above, comprising a LacS transporter sequence having a sequence identity of at least 70% to SEQ ID NO: 1, and/or comprising a GH42 B-gal sequence, wherein the Lactobacillus strain is not a Lactobacillus reuteri strain. Particularly preferably, the probiotic Lactobacillus strain is a Lactobacillus acidophilus (L. acidophilus) strain, more preferably Lactobacillus acidophilus (L. acidophilus) strain NCC12, most preferably a Lactobacillus acidophilus (L. acidophilus) strain NCC12, as deposited under the Budapest treaty under the deposit numbers ATCC-4356 or DSM-20079.
THERAPEUTIC USES OF COMPOSITIONS COMPRISING PRE-CONDITIONED PROBIOTIC LACTOBACILLUS STRAIN AND GOS

According to a fifth aspect, the invention provides a composition comprising an effective amount of a pre-conditioned probiotic Lactobacillus strain supplemented with galacto-oligosaccharides (GOS) as prepared according to the invention or as described herein for use in the prevention and/or treatment of a disease in a patient in need thereof.

As used herein, the terms “treatment,” “treat” and “to alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition, etc. The terms “treatment,” “treat” and “to alleviate” are also intended to include the potentiation or otherwise enhancement of one or more primary prophylactic or therapeutic measure. The terms “treatment,” “treat” and “to alleviate” are further intended to include the dietary management of a disease or condition or the dietary management for prophylaxis or prevention a disease or condition.

A particularly positive effect of the herein used pre-conditioned Lactobacillus strain supplemented with galacto-oligosaccharides (GOS) is an improved microbiota. It is therefore submitted that an increase in survival and/or engraftment and/or viability of a Lactobacillus strain in the intestinal tract of a patient to be treated may specifically and positively influence the treatment of any of the herein-mentioned diseases. The current invention therefore provides as a medical use of the herein described inventive compositions a promotion of immune functions, digestive functions, growth or development, typically in subjects having an imbalance in any of such conditions subjects having deficiencies of such functions or being in need to restore a normal level of such functions or patients being in need of an improvement of any such functions.

The herein described inventive compositions may also be for use in improving or increasing the probiotic effect, preferably of the probiotic Lactobacillus strain in the gastrointestinal tract of the patient.

Such an improving or increasing of the probiotic effect is e.g. a promotion of a healthy microbiota in the intestinal tract of the patient, preferably an increase of the presence of bifidobacteria and/or lactobacilli in the microbiota of the patient; and/or is a promotion of the digestive functions and/or the immune functions and/or growth.

Generally, subject for any such treatments or a patient in need of such a treatment is a mammal, preferably, it is human, more preferably it is selected form a child, a toddler or an infant, an elderly, a pregnant woman, a vegetarian, a lactating woman, a companion animal, but also companion pets such as a cat or a dog, Any of such subjects and patient groups may be treated herein. Such patients may be e.g., at risk of suffering from a digestive dysfunction, an impaired microbiota, and/or has a digestive dysfunction, an impaired microbiota or a condition requiring promotion of the development and/or growth of the microbiota. Also, in this context, any of the methods as discussed before for preparing a composition comprising a pre-conditioned probiotic Lactobacillus strain supplemented with galacto-oligosaccharides (GOS) as well as any of the features of compositions as defined herein comprising the pre-conditioned probiotic Lactobacillus strain also apply mutatis mutandis for the fifth aspect of the current invention, namely to the second medical use of the inventive compositions in any of the herein defined treatments.

This specifically concerns features of the method of preparing a composition comprising a pre-conditioned probiotic Lactobacillus strain supplemented with galacto-oligosaccharides (GOS), and any ingredients and amounts and further features as used for compositions comprising the pre-conditioned probiotic Lactobacillus strain. The composition may be provided in any form as depicted above.

In this context, it is particularly preferred that the inventive composition for second medical uses may be solid or liquid, may be present in form of a powder, a tablet, a capsule, or may be in form of an oil formulation, an emulsion, an oil-in-water emulsion (o/w emulsion), or a water-in oil emulsion (w/o emulsion).

Likewise, the inventive composition for second medical uses may be in an administrable form, preferably selected from the group consisting of nutritional compositions, pharmaceutical formulations, dietary supplements, functional food products, functional beverage products, and combinations thereof, e.g., a dairy product, a milk-based product, a whey protein-based beverage.

As before, the Lactobacillus strain is particularly preferable any Lactobacillus strain known to a skilled person and preferably as defined above, comprising a LacS transporter sequence having a sequence identity of at least 70% to SEQ ID NO: 1, and/or comprising a GH42 B-gal sequence, and wherein the Lactobacillus strain is not a Lactobacillus reuteri strain. Particularly preferably, the probiotic Lactobacillus strain is a Lactobacillus acidophilus (L. acidophilus) strain, more preferably Lactobacillus acidophilus (L. acidophilus) strain NCC12, most preferably a Lactobacillus acidophilus (L. acidophilus) strain NCC12, as deposited under the Budapest treaty under the deposit numbers ATCC-4356 or DSM-20079.

It should be appreciated that the various aspects and embodiments of the detailed description as disclosed herein are illustrative of the specific ways to make and use the invention and do not limit the scope of invention when taken into consideration with the claims and the detailed description. It will also be appreciated that features from aspects and embodiments of the invention may be combined with further features from the same or different aspects and embodiments of the invention.

As used in this detailed description and the appended claims, the singular forms “a,” “an” and “the” include plural referents unless the context clearly dictates otherwise.

All ranges described are intended to include all numbers, whole or fractions, contained within the said range.

### EXAMPLES

### Example 1— Method for Pre-Conditioning L. acidophilus with GOS

Internally produced L. acidophilus NCC12 frozen starter cultures were used to inoculate a fermentation medium containing 4% GOS as carbon source, 4% fructose, 3% yeast extract as nitrogen source, 0.3% tween 80, 1% calcium carbonate powder, 0.001% manganese sulphate and 0.001% magnesium sulphate (% on dry matter). Overnight culture at 37° C. of L. acidophilus was used to inoculate a fresh fermentation medium containing 4% GOS as carbon source, 4% yeast extract as nitrogen source, 0.6% tween 80, 1.5% calcium carbonate powder, 0.0020% manganese sulphate and 0,002% magnesium sulphate (% on dry matter). Fermentation was run at 37° C. and under pH uncontrolled. Standard (not preconditioned) L. acidophilus NCC12 was done by replacing the 4% GOS by 4% dextrose in the fermentation medium. Pre-conditioned L. acidophilus cells were harvested by centrifugation after around 10 h fermentation prior to spray-drying.

Highly purified GOS used as source of GOS was composed of minimum 93% GOS (g/100 g of dry matter).

L. acidophilus cell counts were determined by pour plating. Briefly, serial decimal dilutions spray dried samples were performed in tryptone salt (Oxoid, LP0042) solution. Subsequently, 100 μL of the appropriate dilutions were transferred to petri dishes and mixed with MRS agar (AES Chemunex, Bruz, France). Analysis was performed in duplicate. Plates were then incubated in aerobic conditions at 37° C. for 48 h. Colony forming units per gram (cfu/g) were calculated from the number of colonies counted on plates with appropriate dilution by determining their arithmetic mean.

Furthermore, according to an optional step, GOS was added to the harvested pre-conditioned probiotic L. acidophilus.

### Example 2— Method for Preparing a Composition Comprising Pre-Conditioned L. acidophilus Supplemented with Additional GOS

A cow milk-based toddler beverage, also called growing-up-milk, containing minerals adapted for the age group was prepared by adding an amount of 5×10⁶ cfu per gram pre-conditioned probiotic L. acidophilus and an amount of 0.5 to 10 g GOS/L of conventional commercially available reconstituted growing-up-milk composition.

### Example 3— Composition Comprising Pre-Conditioned L. acidophilus Supplemented with Additional GOS Increases Survival in Colon

a) SHIME model

In this experiment, a simplified simulation of the continuous Simulator of the Human Microbial Ecosystem (SHIME®) was used. This SHIME® model has been extensively used for more than 20 years for both scientific and industrial projects and has been validated with in vivo parameters (see for example Van den Abbeele et al., Arabinoxylo-Oligosaccharides and Inulin Impact Inter-Individual Variation on Microbial Metabolism and Composition, Which Immunomodulates Human cells, J. Agric. Food chem. 2018, 66, 5, 1121-1130).

For the current set of experiments a two-stage batch system mimicking the upper gastro-intestinal tract (Upper GIT, stomach and small intestine) and colonic conditions were used as simplified SHIME® system. Cow milk-based toddler beverage, also called Growing-up-milk, containing minerals adapted for the age group was used in these studies.

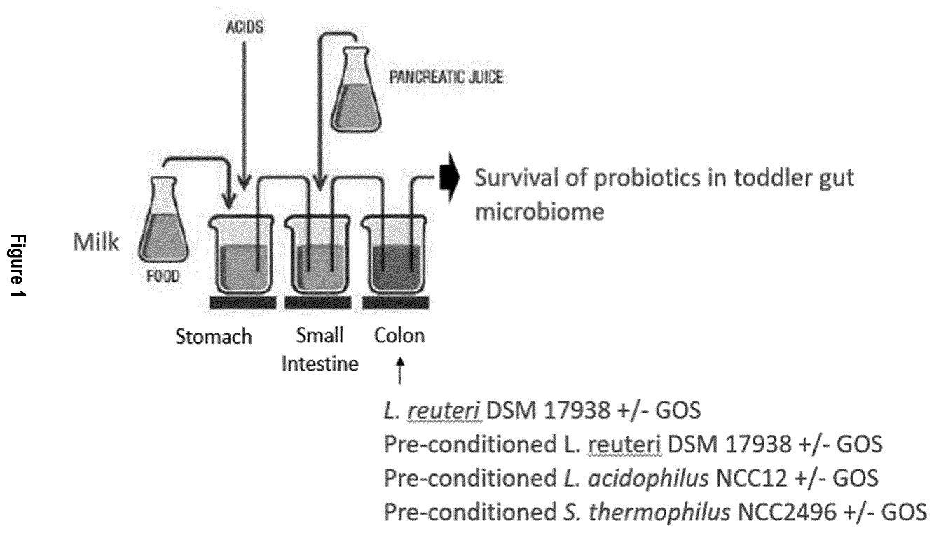

FIG. 1 shows the simplified SHIME® model used in these studies.

In order to simulate the absorptive processes occurring in the small intestine of toddlers, a dialysis approach was applied by using a cellulose membrane with a cut-off of 14 kDa. By introducing the small intestinal suspension within a dialysis membrane, molecules such as digested amino acids, sugars, micronutrients and minerals were gradually removed from the upper gastro-intestinal matrices.

Furthermore, a gradual pH decrease during the stomach incubation going from 5.5 till 3.0 during 1 h of incubation was implemented to simulate the gastric pH of toddlers. Also, during the first 30 minutes of small intestinal incubation (duodenum), a fixed pH of 4.5 was implemented to allow the available minerals to optimally absorb. The following 145 minutes of the small intestinal phase (jejunum+ileum), a pH of 7 was introduced. The milk matrix after exposure to gastric and small intestinal conditions was transferred to the colonic compartment containing the fecal sample of a toddler.

Fresh fecal material was collected from a 12-month-old infant donor. Fecal suspension was prepared and mixed with a protectant. At the start of the short-term colonic incubation, the test ingredients (L. reuteri strain, L. acidophilus strain and Streptococcus thermophilus strain) were added to sugar-depleted nutritional medium containing basal nutrients present in the colon (e.g., host-derived glycans such as mucin).

L. reuteri strain, L. acidophilus strain and S. thermophilus strain were inoculated at 1E+09 CFU/reactor.

Following incubations were performed:
Control incubation without probiotic (Digested milk alone)
Upper GIT digested milk+L. reuteri strain DSM 17938
Upper GIT digested milk+GOS+L. reuteri strain DSM 17938
Upper GIT digested milk+GOS+pre-conditioned L. reuteri strain DSM 17938
Upper GIT digested milk+pre-conditioned S. thermophilus NCC2496
Upper GIT digested milk+GOS+pre-conditioned S. thermophilus NCC2496
Upper GIT digested milk+L. acidophilus NCC12
Upper GIT digested milk+GOS+L. acidophilus NCC12
Upper GIT digested milk+pre-conditioned L. acidophilus NCC12
Upper GIT digested milk+GOS+pre-conditioned L. acidophilus NCC12

Highly purified GOS (>93% purity) was used at equivalent of 4 g GOS/L of digested milk to simulate GOS structures reaching the large intestine.

Internally produced S. thermophilus NCC2496 frozen starter cultures were used to inoculate a fermentation medium containing 3% dextrose as carbon source, 2.5% yeast extract as nitrogen source and 0.1% Tween 80. Overnight culture at 37° C. of S. thermophilus was used to inoculate a fresh fermentation medium containing 4% dextrose as carbon source, 2.5% yeast extract as nitrogen source and 0.1% Tween 80. Fermentation was run at 40° C. and under pH control maintained at 5.8 with addition of NaOH 30%. Preconditioning of S. thermophilus NCC2496 was done by replacing the 3% dextrose by 4% highly purified GOS (>93%) in the fermentation medium.

Internally produced L. acidophilus NCC12 frozen starter cultures were used to inoculate a fermentation medium containing 4% dextrose as carbon source, 4% fructose, 3% yeast extract as nitrogen source, 0.3% Tween 80, 1% calcium carbonate powder, 0.001% manganese sulphate and 0.001% magnesium sulphate (% on dry matter). Overnight culture at 37° C. of L. acidophilus was used to inoculate a fresh fermentation medium containing 4% dextrose as carbon source, 4% yeast extract as nitrogen source, 0.6% Tween 80, 1.5% calcium carbonate powder, 0.0020% manganese sulphate and 0,002% magnesium sulphate (% on dry matter). Fermentation was run at 37° C. and under pH uncontrolled. Preconditioning of L. acidophilus NCC12 was done by replacing the 4% dextrose by 4% highly purified GOS (>93%) in the fermentation medium.

Internally produced L. reuteri frozen starter cultures (L. reuteri DSM 17938) were used to inoculate a fermentation medium containing 4% dextrose as carbon source and 4% fructose as electron acceptor (on dry matter). Overnight culture of L. reuteri was used to inoculate a fresh fermentation medium containing 8% dextrose as carbon source and 8% Fructose as electron acceptor (on dry matter). Fermentation was run under non pH control and at 37° C. Preconditioning of L. reuteri DSM17938 was done by replacing the 8% dextrose by 6% BMOS and 8% Fructose by 6% Fructose. All the other conditions were retained.

S. thermophilus at end of fermentation was plated on M17 supplemented with Lactose and incubated at 37° C. in aerobic for 48 h to estimate CFU/ml.

L. reuteri and L. acidophilus strains were plated on MRS agar and incubated at 37° C. in anaerobic conditions for 48 h to estimate the CFU/ml at the end of fermentation.

Colonic incubations were performed during 48 h, at 37° C. and under shaking (90 rpm) conditions. All experiments were performed in triplicate to account for biological variation.

Microbial community composition qPCR was performed to selectively enumerate Lactobacilli and Streptococci. qPCR was performed at 0 and after 48 hrs of colonic incubation.
b) Genes involved in GOS metabolism

Several genes are known to be involved in GOS metabolism. The transporter LacS and the gene GH42 beta-gal (LacA) are known to be involved in some bacterial strains. L. reuteri DSM17938 has been shown to grow well on BMOS containing GOS. L. reuteri has also been shown to be able to be pre-conditioned. Pre-conditioned L. reuteri has demonstrated stronger survival in short SHIME colonic system.

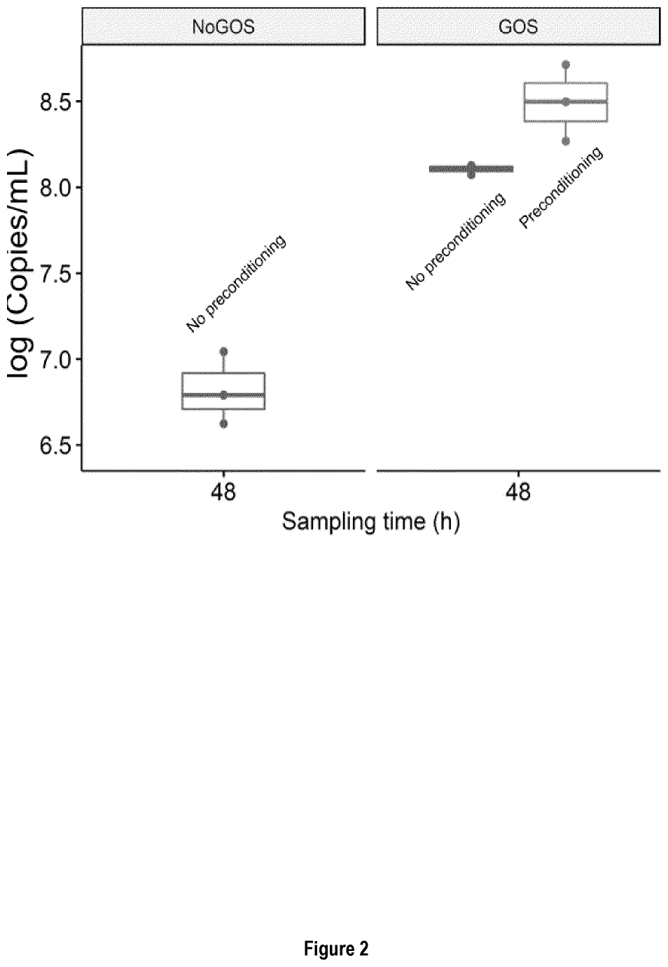

FIG. 2 shows the survival of L. reuteri in presence or absence of GOS in the colon at 48 h. GOS promotes the survival of L. reuteri in the colon. Pre-conditioned L. reuteri exhibits a better survival in presence of GOS compared to the non preconditioned L. reuteri.

Table 1 shows the presence of genes LacS, and LacA in L. acidophilus and S. thermophilus strains as well as the % of sequence homology with L. reuteri DSM17938 sequences. S. thermophilus NCC2496 does not have the gene LacA. LacS gene of S. thermophilus NCC2496 has 36.8% homology with LacS from L. reuteri DSM17938. L. acidophilus NCC12 has the gene LacA being identical to LacA from L. reuteri at 70%. LacS of L. acidophilus NCC12 is identical to L. reuteri at 70%. Hence the main difference between the 3 strains is the absence of GH42 beta-gal (LacA) in S. thermophilus NCC2496. L. acidophilus NCC12 is equipped with LacS and LacA. LacS and LacA having more than 70% homology with L. reuteri DSM17938 sequences.

TABLE 1
Genes involved in GOS metabolism

| | Transporter (LacS) | | | GH43 B-gal (LacA) | | |
|---|---|---|---|---|---|---|
| | | | Length | | | Length |
| | ID | % ID | hit(bp) | ID | % ID | hit(bp) |
| L. reuteri DSM 17983 | CDS0114 | 100 | 640 | CDS0115 | 100 | 673 |
| L. acidophilus NCC12 | CDS1417 | 70.938 | 640 | CDS1416 | 70.827 | 665 |
| S. thermophilus NCC2496 | CDS1409 | 36.83 | 611 | #N/A | #N/A | #N/A |

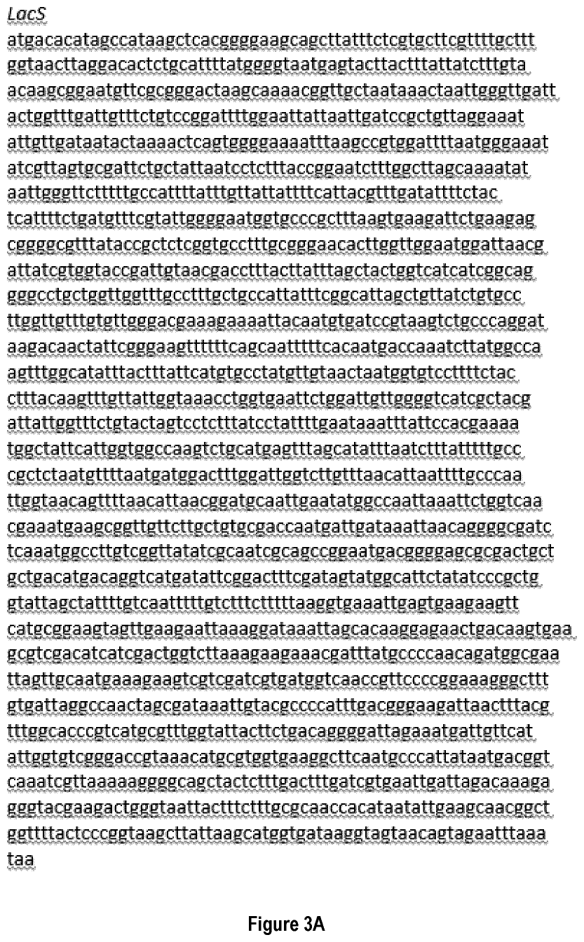

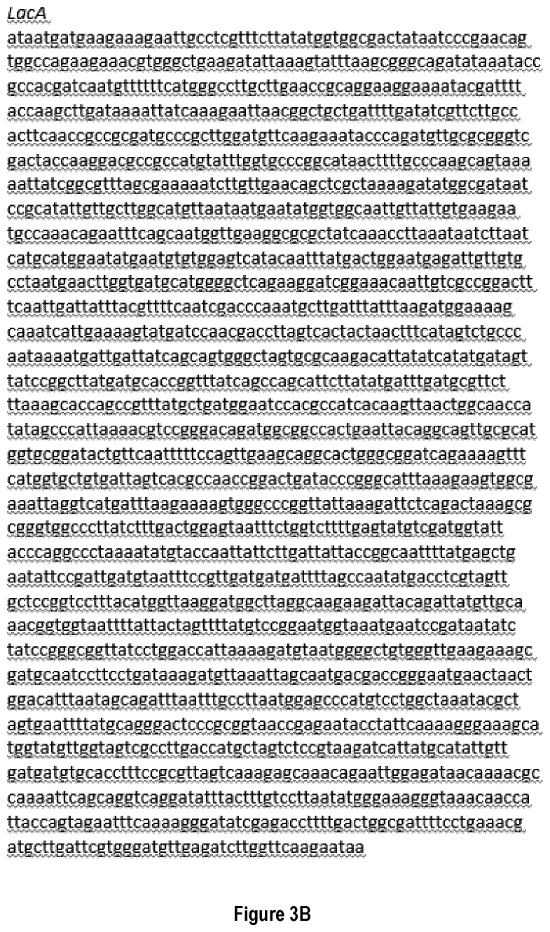

FIGS. 3A and 3B show the corresponding LacS sequence (SEQ ID NO: 1) and LacA sequence (SEQ ID NO: 2) of L. reuteri DSM 17938.
c) Activation of the beta-galactosidase enzyme

The beta-galactosidase enzyme is likely to be activated in bacteria grown on GOS, BMOS or lactose. Samples of L. acidophilus NCC12 and S. thermophilus NCC2496 were taken at the end of the fermentation on dextrose (reference) or GOS (preconditioning). Samples were washed twice with PBS by centrifugation 5 min at 5000 g. Pellets were resuspended in PBS to 1E+8 AFU/mL for L. acidophilus and 1E+7 AFU/mL for S. thermophilus. For that purpose, AFU were determined by flow cytometry.

X-Gal (Thermo Scientific, B1690) was added to the samples at a final concentration of 0.77 mg/mL.

The enzymatic conversion of the X-gal into a blue product was followed through OD measurement at 420 and 670 nm in a spectrophotometer over a period of 2 h at 37° C. The beta-galactosidase activity was estimated by measuring the slope of the enzymatic conversion.

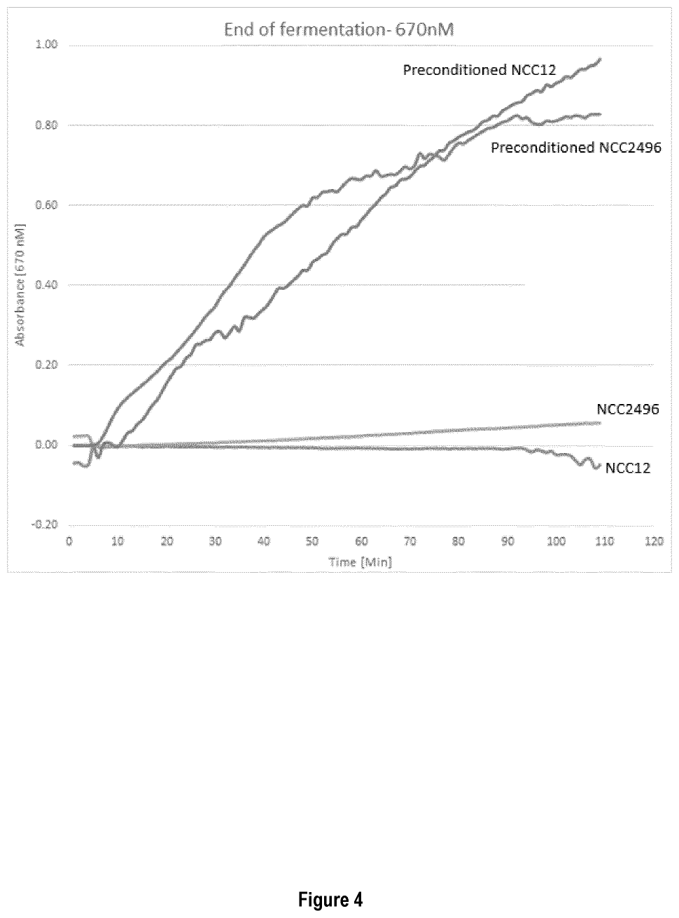

FIG. 4 shows the beta-galactosidase activity in L. acidophilus NCC12 and S. thermophilus NCC2496 and their pre-conditioned counterparts. Both preconditioned L. acidophilus NCC12 and preconditioned S. thermophilus NCC2496 strains exhibit a strong beta-galactosidase activity, shown by a constant conversion of X-gal substrate over the 2 h incubation, compared to their control strains grown on dextrose.

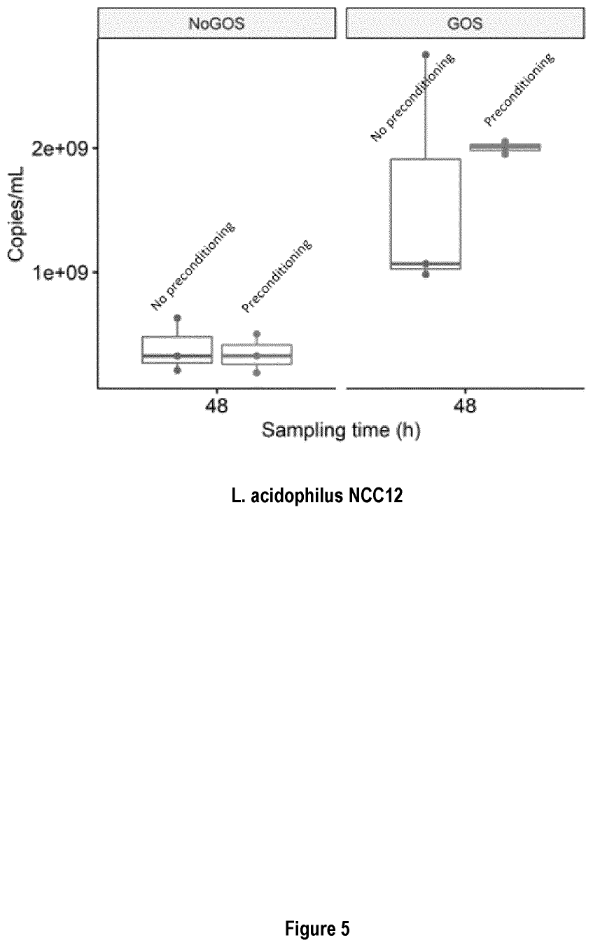

FIG. 5 shows the viability of L. acidophilus NCC12 (pre-conditioned and not pre-conditioned) in the colon in presence and absence of GOS at 48 h incubation. GOS promotes the survival of L. acidophilus NCC12 regardless the source of carbon to grow the strain. Both preconditioned (GOS grown) and non pre-conditioned (dextrose grown) L. acidophilus NCC12 survive better in the colon in presence of GOS. Preconditioned L. acidophilus NCC12 survives better in the colon in presence of GOS than its counterpart grown on dextrose, showing the positive effect of the preconditioning step on the survival of L. acidophilus NCC12 in the colon in presence of GOS.

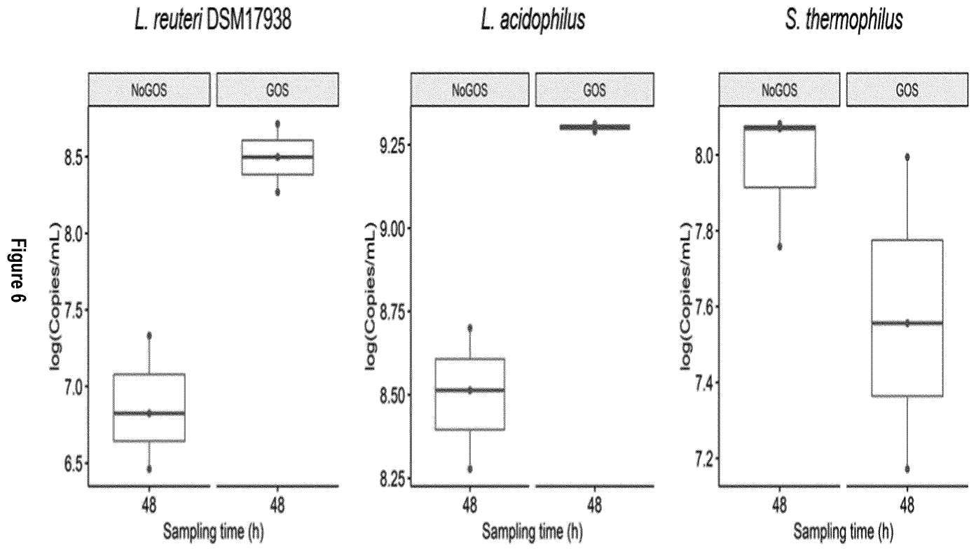

FIG. 6 shows the survival of L. reuteri DSM17938, L. acidophilus NCC12 and Streptococcus thermophilus NCC2496 in presence or absence of GOS in the colon after 48 h incubation, particularly a positive effect of GOS on L. reuteri and L. acidophilus NCC12 survival and negative effect of GOS on Streptococcus thermophilus NCC2496 survival. While both L. acidophilus and S. thermophilus strains grow on GOS and exhibit strong beta-galactosidase activity (FIG. 4), only preconditioned L. acidophilus survives better in the colon in presence of GOS. S. thermophilus happens to miss Lac A in the genome. Pre-conditioned L. acidophilus survives well in the colon as also observed with pre-conditioned L. reuteri. Both L. reuteri and L. acidophilus have LacA gene, combined with high homology sequences for LacS (>70%). This means that presence of LacA gene combined with LacS having a sequence identity of at least 70% to the sequence found in L. reuteri DSM17938 is a feature that helps pre-conditioned strains surviving in the gut in presence of GOS.

1. A method for preparing a composition comprising a pre-conditioned probiotic Lactobacillus strain and GOS, the method comprises the steps:
a) pre-conditioning of a probiotic Lactobacillus strain following the steps:
i) cultivating a probiotic Lactobacillus strain in the presence of galacto-oligosaccharide (GOS) in a growth medium, thereby pre-conditioning the probiotic Lactobacillus strain; and
b) preparing a composition comprising the pre-conditioned probiotic Lactobacillus strain obtained and adding GOS to the composition,
wherein the probiotic Lactobacillus strain is at least one Lactobacillus strain comprising a LacS transporter sequence having a sequence identity of at least 70% to SEQ ID NO: 1, and/or comprising a GH42 B-gal sequence, wherein the Lactobacillus strain is not a Lactobacillus reuteri strain.

2. The method according to claim 1, wherein GOS is added in step a)i) to the growth medium in an amount of at least 0.2 wt % calculated on basis of the growth medium (% w/w).

3. The method according to claim 1, wherein GOS is added in to the pre-conditioned probiotic Lactobacillus strain in an amount of at least 1 or 2 g per daily dose.

4. The method according to claim 1, wherein the pre-conditioned probiotic Lactobacillus strain obtained is present in the composition in an amount of between 10³ cfu to 10¹² cfu per daily dose.

5. The method according to claim 1, wherein pH during cultivation is controlled.

6. The method according to claim 1, wherein the method for preparing a pre-conditioned probiotic Lactobacillus strain comprises
a further step of washing the pre-conditioned probiotic Lactobacillus strain after harvesting step, and
drying the harvested pre-conditioned probiotic Lactobacillus strain.

7. The method according to claim 1, wherein the probiotic Lactobacillus strain is a Lactobacillus acidophilus (L. acidophilus) strain.

8. A composition comprising:
an effective amount of a pre-conditioned probiotic Lactobacillus strain, and
(ii) an effective amount of a galacto-oligosaccharide (GOS), and
wherein the probiotic Lactobacillus strain is provided from at least one Lactobacillus strain comprising a LacS transporter sequence having a sequence identity of at least 70% to SEQ ID NO: 1, and/or comprising a GH42 B-gal sequence, wherein the Lactobacillus strain is not a Lactobacillus reuteri strain.

9. (canceled)

10. The composition according to claim 8,
wherein the composition is an administrable form, selected from the group consisting of nutritional compositions, pharmaceutical formulations, dietary supplements, functional food products, functional beverage products, a nutritional composition for infant or children such as an infant formula or follow-on formula, and combinations thereof.

11. Method for improving or increasing the probiotic effect of a probiotic Lactobacillus strain in a healthy subject,
comprising administering a composition comprising an effective amount of a pre-conditioned probiotic Lactobacillus strain supplemented with an effective amount of GOS,
wherein improving or increasing the probiotic effect:
is promoting the survival or metabolic activity of the Lactobacillus strain in the colon of the healthy subject and thus a promotion of the health benefits associated to the Lactobacillus strains; and
promotion of a healthy microbiota in the intestinal tract of the healthy subject.

12. Method according to claim 11, wherein the healthy subject is selected from the group consisting of an athlete, a child, a toddler or an infant, an adult, an elderly, a pregnant woman, a vegetarian, a lactating woman, a companion animal, a cat, and a dog.

13. The non-therapeutic use according to claim 11,
wherein the composition is an administrable form, selected from the group consisting of nutritional compositions, pharmaceutical formulations, dietary supplements, functional food products, functional beverage products, nutritional compositions for infants or children, and combinations thereof.

14-15. (canceled)


SEQUENCE LISTING (2 sequences)

SEQ ID NO: 1 - DNA, 1923 bp, Lactobacillus
  >LACRE_NCC3025_CDS0114 Gene= 4126 6049 Direct Product=Glycoside/pentoside/hexuronide transporter {ECO0000313|EMBLAEI56355.1
atgacacatagccataagctcacggggaagcagcttatttctcgtgcttcgttttgctttggtaacttaggacactctgcattttatggggtaatgagtacttactttattatctttgtaacaagcggaatgttcgcgggactaagcaaaacggttgctaataaactaattgggttgattactggtttgattgtttctgtccggattttggaattattaattgatccgctgttaggaaatattgttgataatactaaaactcagtggggaaaatttaagccgtggattttaatgggaaatatcgttagtgcgattctgctattaatcctctttaccggaatctttggcttagcaaaatataattgggttctttttgccattttatttgttattattttcattacgtttgatattttctactcattttctgatgtttcgtattggggaatggtgcccgctttaagtgaagattctgaagagcggggcgtttataccgctctcggtgcctttgcgggaacacttggttggaatggattaacgattatcgtggtaccgattgtaacgacctttacttatttagctactggtcatcatcggcaggggcctgctggttggtttgcctttgctgccattatttcggcattagctgttatctgtgccttggttgtttgtgttgggacgaaagaaaattacaatgtgatccgtaagtctgcccaggataagacaactattcgggaagttttttcagcaatttttcacaatgaccaaatcttatggccaagtttggcatatttactttattcatgtgcctatgttgtaactaatggtgtccttttctacctttacaagtttgttattggtaaacctggtgaattctggattgttggggtcatcgctacgattattggtttctgtactagtcctctttatcctattttgaataaatttattccacgaaaatggctattcattggtggccaagtctgcatgagtttagcatatttaatctttatttttgcccgctctaatgttttaatgatggactttggattggtcttgtttaacattaattttgcccaattggtaacagttttaacattaacggatgcaattgaatatggccaattaaattctggtcaacgaaatgaagcggttgttcttgctgtgcgaccaatgattgataaattaacaggggcgatctcaaatggccttgtcggttatatcgcaatcgcagccggaatgacggggagcgcgactgctgctgacatgacaggtcatgatattcggactttcgatagtatggcattctatatcccgctggtattagctattttgtcaatttttgtctttctttttaaggtgaaattgagtgaagaagttcatgcggaagtagttgaagaattaaaggataaattagcacaaggagaactgacaagtgaagcgtcgacatcatcgactggtcttaaagaagaaacgatttatgccccaacagatggcgaattagttgcaatgaaagaagtcgtcgatcgtgatggtcaaccgttccccggaaagggctttgtgattaggccaactagcgataaattgtacgccccatttgacgggaagattaactttacgtttggcacccgtcatgcgtttggtattacttctgacaggggattagaaatgattgttcatattggtgtcgggaccgtaaacatgcgtggtgaaggcttcaatgcccattataatgacggtcaaatcgttaaaaaggggcagctactctttgactttgatcgtgaattgattagacaaagagggtacgaagactgggtaattactttctttgcgcaaccacataatattgaagcaacggctggttttactcccggtaagcttattaagcatggtgataaggtagtaacagtagaatttaaataa

SEQ ID NO: 2 - DNA, 2022 bp, Lactobacillus
  >LACRE_NCC3025_CDS0115 Gene= 2098 4120 Direct Product=Beta-galactosidase  Beta-galactosidase {ECO0000256|PIRNRPIRNR001084; Beta-gal {ECO0000256|PIRNRPIRNR001084; EC=3.2.1.23 {ECO0000256|PIRNRPIRNR001084
ataatgatgaagaaagaattgcctcgtttcttatatggtggcgactataatcccgaacagtggccagaagaaacgtgggctgaagatattaaagtatttaagcgggcagatataaataccgccacgatcaatgttttttcatgggccttgcttgaaccgcaggaaggaaaatacgattttaccaagcttgataaaattatcaaagaattaacggctgctgattttgatatcgttcttgccacttcaaccgccgcgatgcccgcttggatgttcaagaaatacccagatgttgcgcgggtcgactaccaaggacgccgccatgtatttggtgcccggcataacttttgcccaagcagtaaaaattatcggcgtttagcgaaaaatcttgttgaacagctcgctaaaagatatggcgataatccgcatattgttgcttggcatgttaataatgaatatggtggcaattgttattgtgaagaatgccaaacagaatttcagcaatggttgaaggcgcgctatcaaaccttaaataatcttaatcatgcatggaatatgaatgtgtggagtcatacaatttatgactggaatgagattgttgtgcctaatgaacttggtgatgcatggggctcagaaggatcggaaacaattgtcgccggactttcaattgattatttacgttttcaatcgacccaaatgcttgatttatttaagatggaaaagcaaatcattgaaaagtatgatccaacgaccttagtcactactaactttcatagtctgcccaataaaatgattgattatcagcagtgggctagtgcgcaagacattatatcatatgatagttatccggcttatgatgcaccggtttatcagccagcattcttatatgatttgatgcgttctttaaagcaccagccgtttatgctgatggaatccacgccatcacaagttaactggcaaccatatagcccattaaaacgtccgggacagatggcggccactgaattacaggcagttgcgcatggtgcggatactgttcaatttttccagttgaagcaggcactgggcggatcagaaaagtttcatggtgctgtgattagtcacgccaaccggactgatacccgggcatttaaagaagtggcgaaattaggtcatgatttaagaaaagtgggcccggttattaaagattctcagactaaagcgcgggtggcccttatctttgactggagtaatttctggtcttttgagtatgtcgatggtattacccaggccctaaaatatgtaccaattattcttgattattaccggcaattttatgagctgaatattccgattgatgtaatttccgttgatgatgattttagccaatatgacctcgtagttgctccggtcctttacatggttaaggatggcttaggcaagaagattacagattatgttgcaaacggtggtaattttattactagttttatgtccggaatggtaaatgaatccgataatatctatccgggcggttatcctggaccattaaaagatgtaatggggctgtgggttgaagaaagcgatgcaatccttcctgataaagatgttaaattagcaatgacgaccgggaatgaactaactggacatttaatagcagatttaatttgccttaatggagcccatgtcctggctaaatacgctagtgaattttatgcagggactcccgcggtaaccgagaatacctattcaaaagggaaagcatggtatgttggtagtcgccttgaccatgctagtctccgtaagatcattatgcatattgttgatgatgtgcacctttccgcgttagtcaaagagcaaacagaattggagataacaaaacgccaaaattcagcaggtcaggatatttactttgtccttaatatgggaaagggtaaacaaccattaccagtagaatttcaaaagggatatcgagaccttttgactggcgattttcctgaaacgatgcttgattcgtgggatgttgagatcttggttcaagaataa